(19) United States
(12) Patent Application Publication
(10) Pub. No.: US 2025/0369037 A1
(43) Pub. Date: Dec. 4, 2025

(54) METHODS, COMPOSITIONS, AND KITS FOR SPATIAL DETECTION OF TARGET NUCLEIC ACIDS

(71) Applicant: 10x Genomics, Inc., Pleasanton, CA (US)

(72) Inventor: Anushka Gupta, Milpitas, CA (US)

(21) Appl. No.: 19/228,286

(22) Filed: Jun. 4, 2025

Related U.S. Application Data
(60) Provisional application No. 63/655,740, filed on Jun. 4, 2024.

(51) Int. Cl. C12Q 1/6841; C12Q 1/6823

(52) U.S. Cl. CPC C12Q 1/6841; C12Q 1/6823

(57) ABSTRACT

Provided herein are methods, compositions, and kits for the transposome-mediated capture of analytes on a substrate including spatially barcoded capture probes.

### CROSS-REFERENCE TO RELATED APPLICATIONS

This application claims the benefit of priority to U.S. Provisional Patent Application No. 63/655,740 filed on Jun. 4, 2024, the contents of which are hereby incorporated by reference.

### SEQUENCE LISTING

This application contains a Sequence Listing that has been submitted electronically as an XML file named “47706-0392001_SL_ST26.XML.” The XML file, created on May 30, 2025, is 32,243 bytes in size. The material in the XML file is hereby incorporated by reference in its entirety.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling and cross-talk with other cells in the tissue.

Spatial heterogeneity has been previously studied using techniques that only provide data for a small handful of analytes in the context of an intact tissue or a portion of a tissue, or provide substantial analyte data for dissociated tissue (i.e., single cells), but fail to provide information regarding the position of the single cell in a parent biological sample (e.g., tissue sample).

Various types of tissues often require different treatment conditions, e.g., permeabilization conditions such as the use of different enzymes, duration, and additional reagents (e.g., detergents, surfactants), etc., when performing spatial analyses based on factors such as extracellular matrix proteins among others. Resources such as reagents, time, and sequencing costs can be wasted when determining optimal conditions for spatial analysis.

Improved methods and conditions are still needed, which can be applied across various tissue types without excessive experimental testing.

### SUMMARY

The present disclosure features methods, compositions, and kits to determine the location of target nucleic acids in a biological sample. The methods described herein can be used across various species and/or types of tissue, thereby foregoing the need for tissue specific permeabilization optimization. For example, human breast cancer is typically rich in collagenase and can require additional permeabilization condition testing to determine optimal conditions to perform spatial transcriptomic analyses. Thus, the disclosed methods, kits and compositions can be useful in performing spatial analysis without the need for tissue permeabilization optimization. The present disclosure utilizes, in some embodiments, an in situ reverse transcription, followed by tagmentation, and capture of the fragmented products on a spatial array as further described herein.

Thus provided herein are methods for determining a location of a target RNA in a biological sample, the method including: a) hybridizing a primer to the target RNA in the biological sample; b) extending the primer using the target RNA as a template to provide a cDNA hybridized to the target RNA, thereby generating a cDNA:RNA duplex; c) incorporating at least three untemplated nucleotides at a 3′ end of the cDNA of the cDNA:RNA duplex; d) hybridizing a first adapter to the at least three untemplated nucleotides and extending the cDNA of the cDNA:RNA duplex using the first adapter as a template, thereby generating an extended cDNA:RNA duplex; e) contacting a transposome complex with the biological sample to insert a second adapter into the extended cDNA:RNA duplex, thereby generating a 5′ fragmented cDNA:RNA duplex; f) releasing the RNA from the 5′ fragmented cDNA:RNA duplex, thereby generating a 5′ cDNA molecule including (i) a complement of the first adapter, and (ii) the second adapter; g) hybridizing the first adapter of the 5′ cDNA molecule to a capture domain of a capture probe in an array including a plurality of capture probes, where the capture probe includes: (i) a spatial barcode and (ii) a capture domain; and h) determining the sequence of (i) the spatial barcode, or a complement thereof, (ii) the 5′ cDNA molecule or a complement thereof, and using the determined sequences of (i) and (ii) to determine the location of the target RNA in the biological sample.

In some embodiments, the biological sample is disposed on the array or where the biological sample is disposed on a first substrate. In some embodiments, when the biological sample is disposed on a first substrate, the method includes aligning the first substrate including the biological sample with a second substrate including the array, such that at least a portion of the biological sample is aligned with at least a portion of the array, and migrating the 5′ cDNA molecule from the biological sample to the array, optionally where the migrating includes electrophoresis.

In some embodiments, the extending in step (b) includes use of a reverse transcriptase, where incorporating the at least three untemplated nucleotides includes use of the reverse transcriptase. In some embodiments, incorporating the at least three untemplated nucleotides includes use of a terminal transferase, optionally where the terminal transferase is a terminal deoxynucleotidyl transferase.

In some embodiments, the first adapter includes RNA.

In some embodiments, the at least three untemplated nucleotides include a homopolynucleotide sequence or a heteropolynucleotide sequence.

In some embodiments, the second adapter sequences include a functional domain, optionally where the functional domain includes a primer binding site.

In some embodiments, the transposome complex includes a transposase enzyme, a transposon sequence, and the second adapter; and optionally where the transposase enzyme is a Tn5 transposase enzyme, a Mu transposase enzyme, a Tn7 transposase enzyme, a Vibrio species transposase, or functional derivatives thereof.

In some embodiments, releasing the RNA includes use of heat, potassium hydroxide, or an RNase, optionally where the RNase include one or more of RNase A, RNase C, RNase H, and RNase I.

In some embodiments, the second adapter is inserted at a 5′ end of the cDNA in the 5′ fragmented cDNA:RNA duplex.

In some embodiments, step (e) includes generating a 3′ fragmented cDNA:RNA duplex and one or more middle fragmented cDNA:RNA duplexes, the method including a reverse transcription reaction to gap-fill the 3′ fragmented cDNA:RNA duplex and the one or more middle fragmented cDNA:RNA duplexes.

In some embodiments, releasing the RNA from the 3′ fragmented cDNA:RNA duplex and the one or more middle fragmented cDNA:RNA duplexes, thereby generating a 3′ cDNA molecule and one or more middle cDNA molecule(s), respectively.

In some embodiments, the array includes a second plurality of capture probes, where a second capture probe of the second plurality of capture probes includes: (i) a second spatial barcode and (ii) a second capture domain; where the second adapter includes a sequence complementary to the second capture domain.

In some embodiments, the method includes hybridizing the second adapter of the 3′ cDNA molecule to the second capture domain, and determining the sequence of: (i) the second spatial barcode, or a complement thereof, and (ii) the 3′ cDNA molecule, or a complement thereof, and using the determined sequences of (i) and (ii) to determine the location of the target RNA in the biological sample.

In some embodiments, the method includes extending the capture probe using the 5′ cDNA molecule as a template and/or extending the 5′ cDNA molecule using the capture probe as a template.

In some embodiments, the determining step includes sequencing.

In some embodiments, the method includes permeabilizing, staining, and/or imaging the biological sample, and where the biological sample is a tissue section, optionally a fresh-frozen tissue section or a fixed tissue section.

In some embodiments, the target RNA is mRNA.

Also provided herein are methods for processing a target RNA in a biological sample, the method including: a) hybridizing a primer to the target RNA in the biological sample; b) extending the primer using the target RNA as a template to provide a cDNA hybridized to the target RNA, thereby generating a cDNA:RNA duplex; c) incorporating at least three untemplated nucleotides at a 3′ end of the cDNA of the cDNA:RNA duplex; d) hybridizing a first adapter to the at least three untemplated nucleotides and extending the cDNA of the cDNA:RNA duplex using the first adapter as a template, thereby generating an extended cDNA:RNA duplex; e) contacting a transposome complex with the biological sample to insert a second adapter into the extended cDNA:RNA duplex, thereby generating a 5′ fragmented cDNA:RNA duplex; and f) releasing the RNA from the 5′ fragmented cDNA:RNA duplex, thereby generating a 5′ cDNA molecule including a complement of the first adapter and the second adapter.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “about” or “approximately” as used herein means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “substantially complementary” used herein means that a first sequence is at least 50%, 55%, 60%, 65%, 70%, 75%, 80%, 85%, 90%, 95%, 97%, 98% or 99% identical to the complement of a second sequence over a region of 2, 3, 4, 5, 6, 7, 8, 9, 10, 11, 12, 13, 14, 15, 16, 17, 18, 19, 20-40, 40-60, 60-100, or more nucleotides, or that the two sequences hybridize under stringent hybridization conditions. Substantially complementary also means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations known to those skilled in the art.

The term “each,” when used in reference to a collection of items, is intended to identify an individual item in the collection but does not necessarily refer to every item in the collection, unless expressly stated otherwise, or unless the context of the usage clearly indicates otherwise. Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

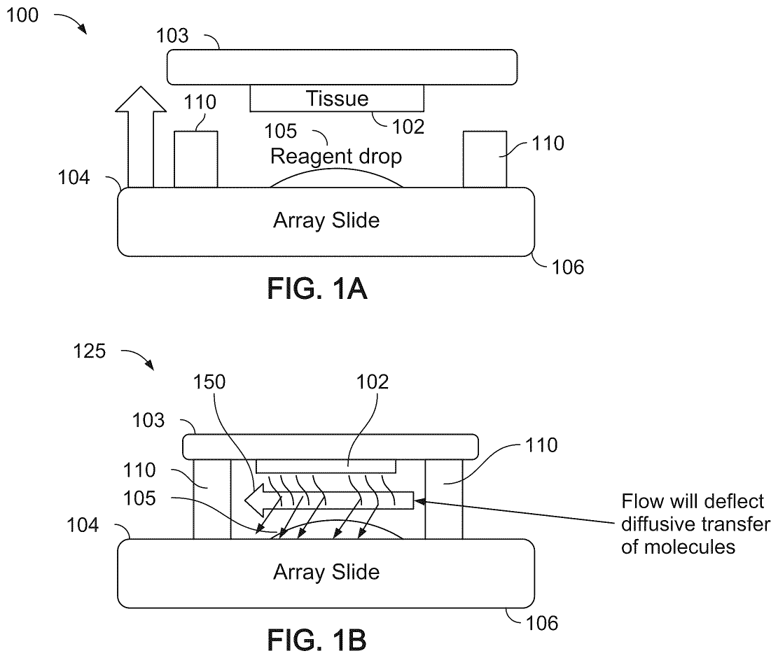

FIG. 1A shows an exemplary sandwiching process where a first substrate (e.g., a slide), including a biological sample, and a second substrate (e.g., array slide) are brought into proximity with one another.

FIG. 1B shows a fully formed sandwich configuration creating a chamber formed from the one or more spacers, the first substrate, and the second substrate.

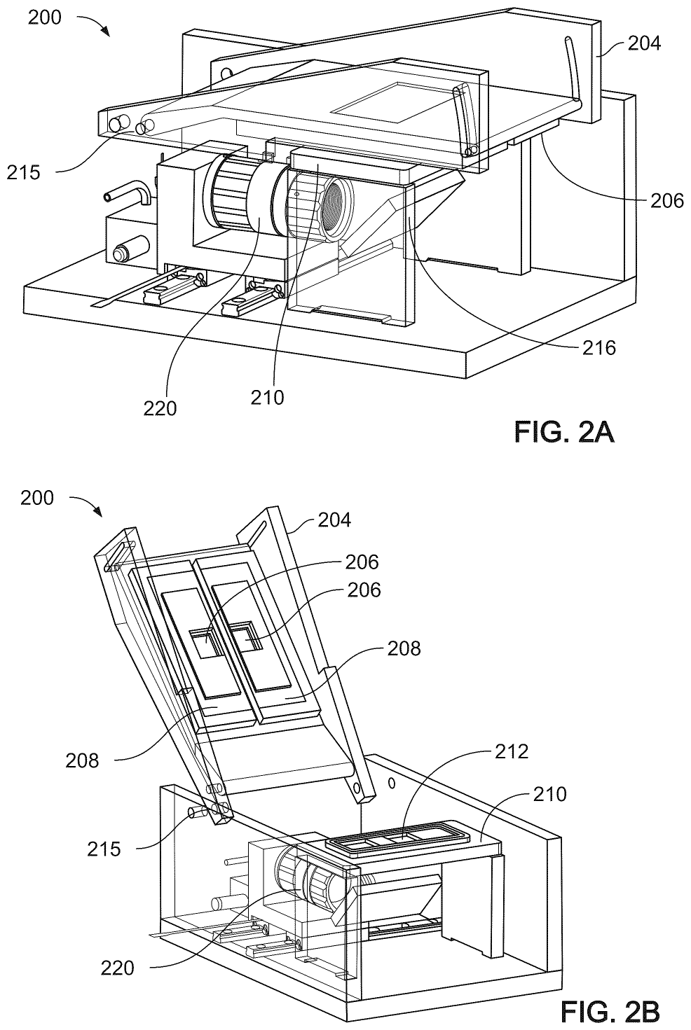

FIG. 2A shows a perspective view of an exemplary sample handling apparatus in a closed position.

FIG. 2B shows a perspective view of an exemplary sample handling apparatus in an open position.

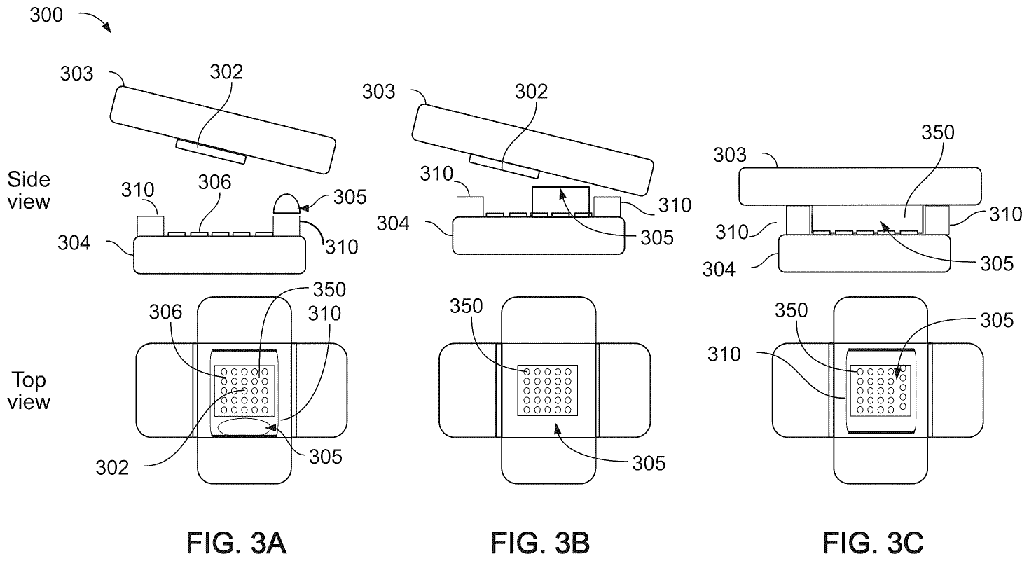

FIG. 3A shows the first substrate angled over (superior to) the second substrate.

FIG. 3B shows that as the first substrate lowers, and/or as the second substrate rises, the dropped side of the first substrate may contact a drop of reagent medium.

FIG. 3C shows a full closure of the sandwich between the first substrate and the second substrate with one or more spacers contacting both the first substrate and the second substrate.

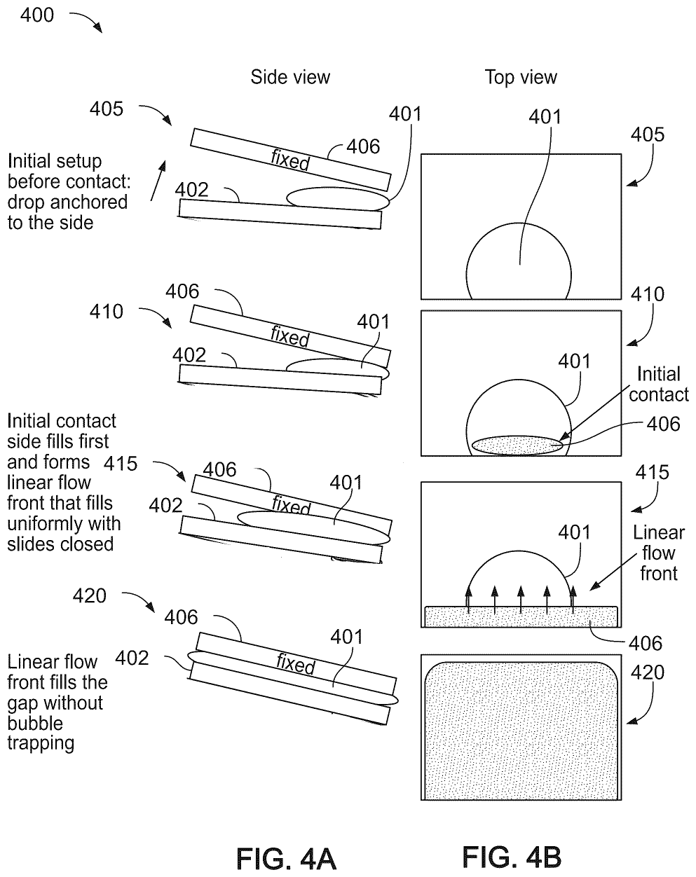

FIG. 4A shows a side view of the angled closure workflow.

FIG. 4B shows a top view of the angled closure workflow.

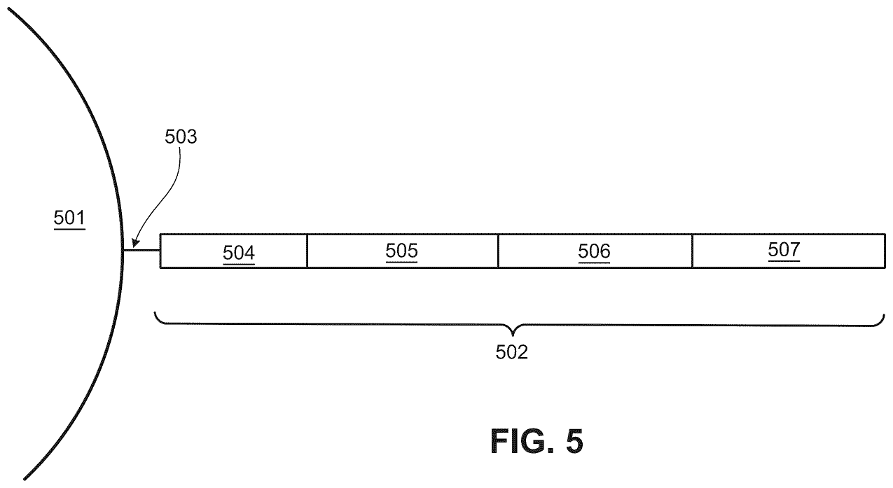

FIG. 5 is a schematic diagram showing an example of a barcoded capture probe, as described herein.

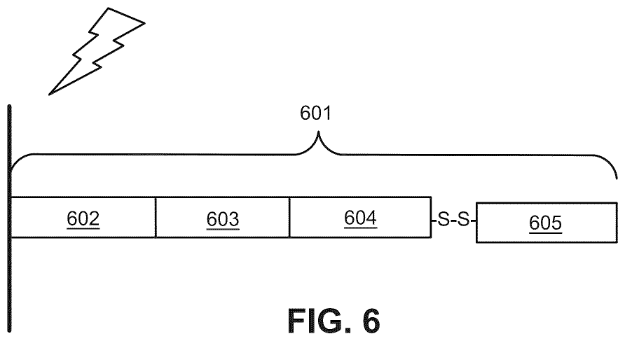

FIG. 6 shows a schematic illustrating a cleavable capture probe.

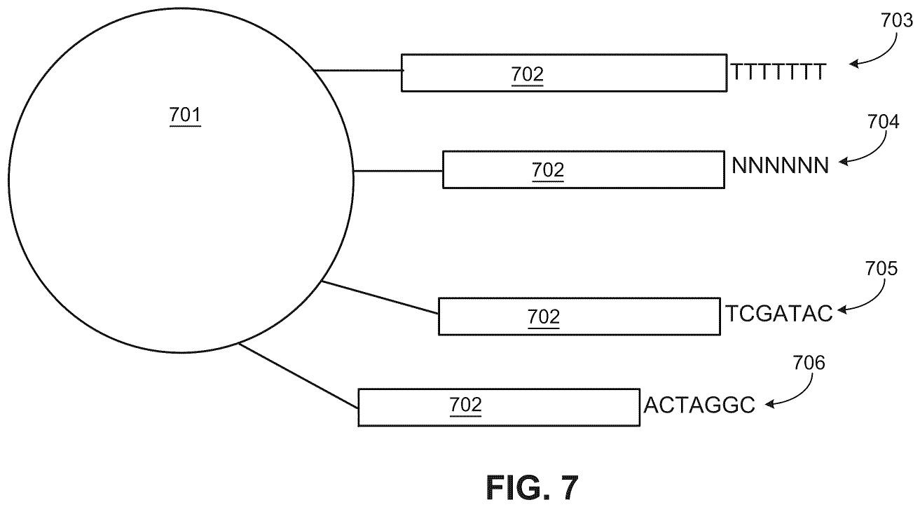

FIG. 7 shows exemplary capture domains on capture probes.

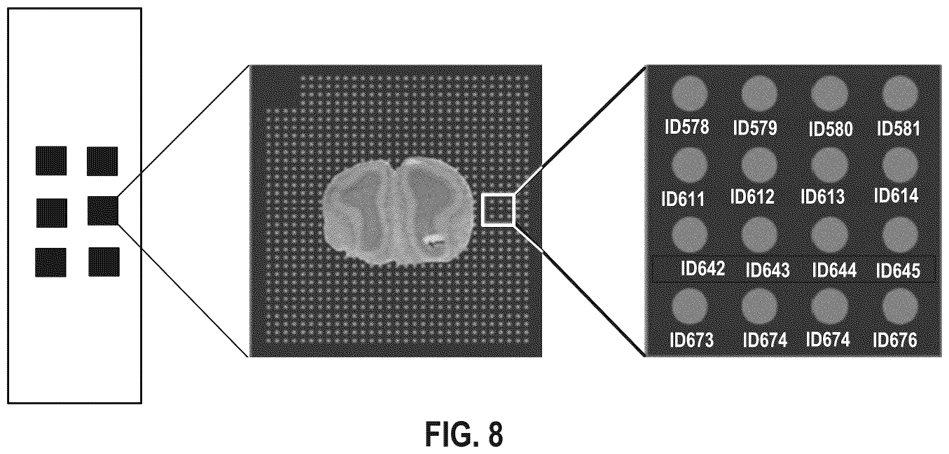

FIG. 8 shows an exemplary arrangement of barcoded features within an array.

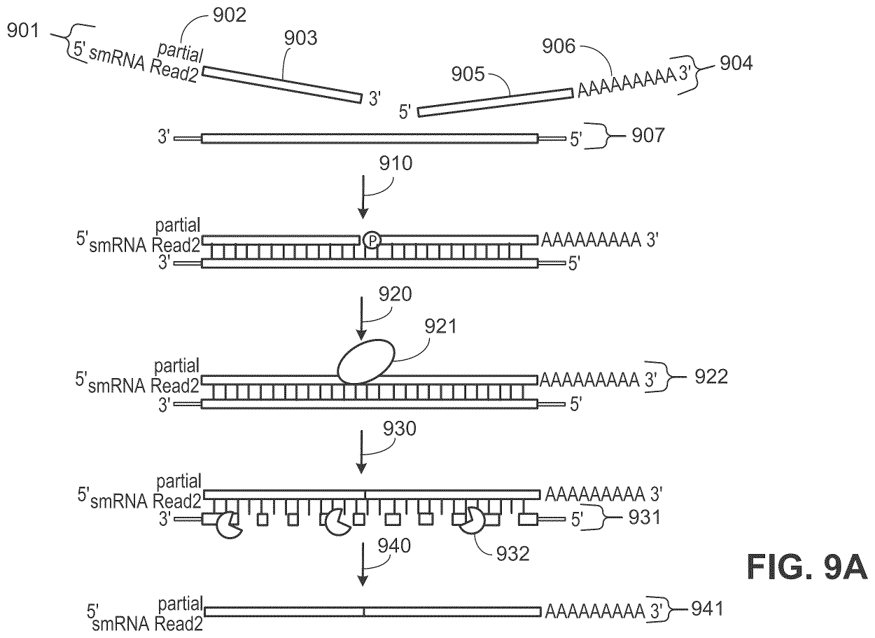

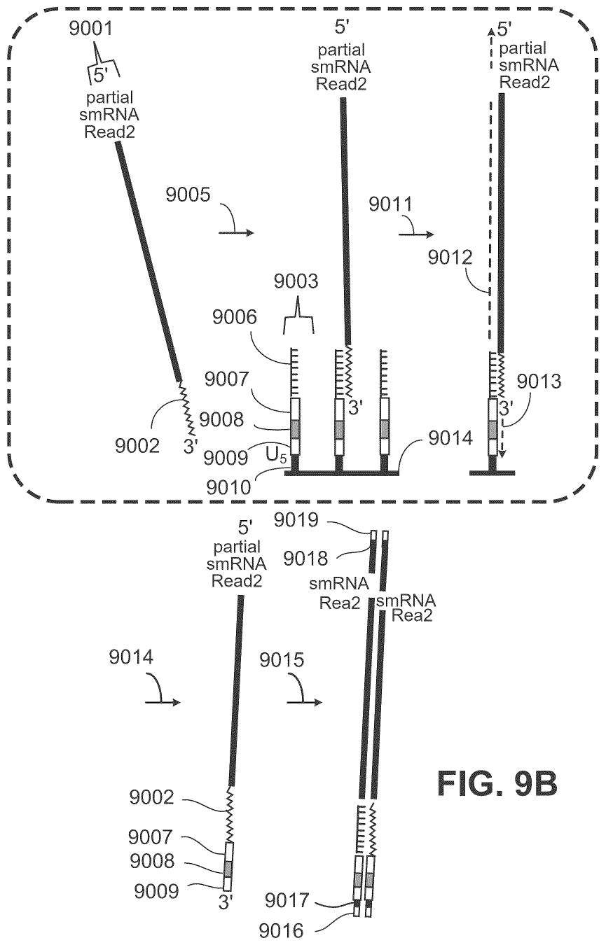

FIG. 9A shows and exemplary workflow for performing templated capture and producing a ligation product, and FIG. 9B shows an exemplary workflow for capturing a ligation product from FIG. 9A on a substrate.

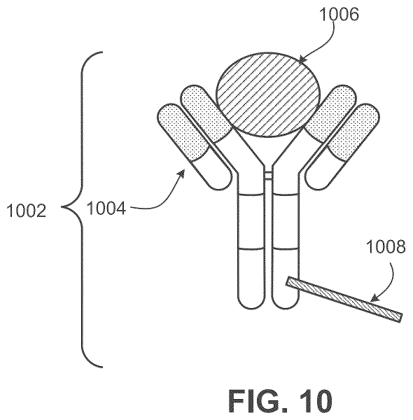

FIG. 10 is a schematic diagram of an exemplary analyte capture agent.

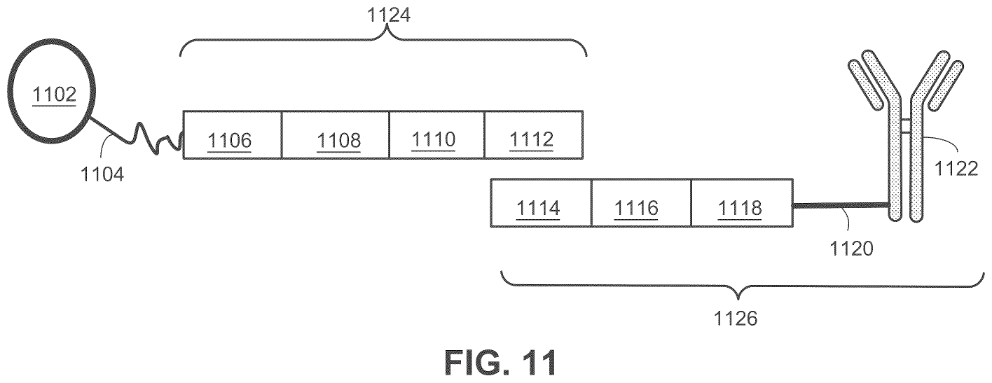

FIG. 11 is a schematic diagram depicting an exemplary interaction between a feature-immobilized capture probe 1124 and an analyte capture agent 1126.

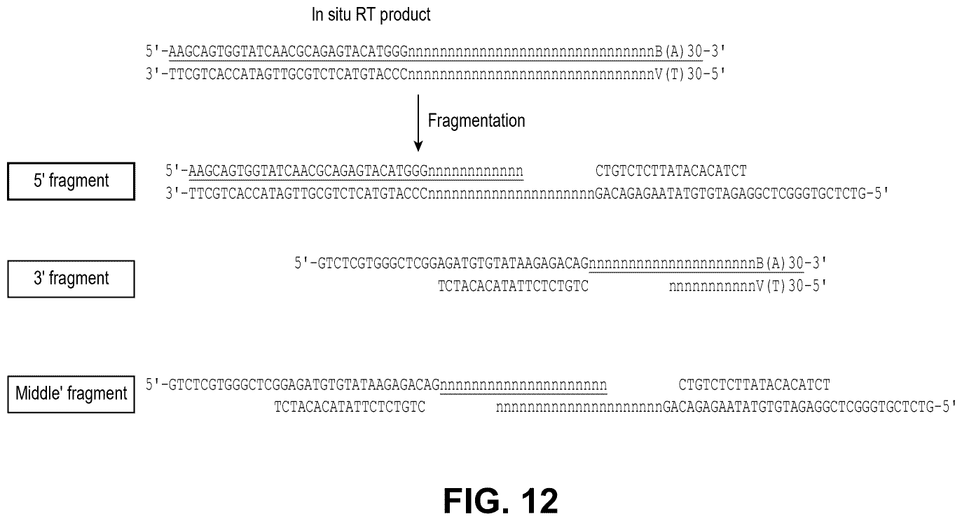

FIG. 12 from top to bottom is a schematic showing an example of an in situ reverse transcribed cDNA product hybridized to a target RNA (top) followed by three different cDNA:RNA fragments (e.g., a 5′ fragment, a 3′ fragment, and a middle fragment) generated after tagmentation.

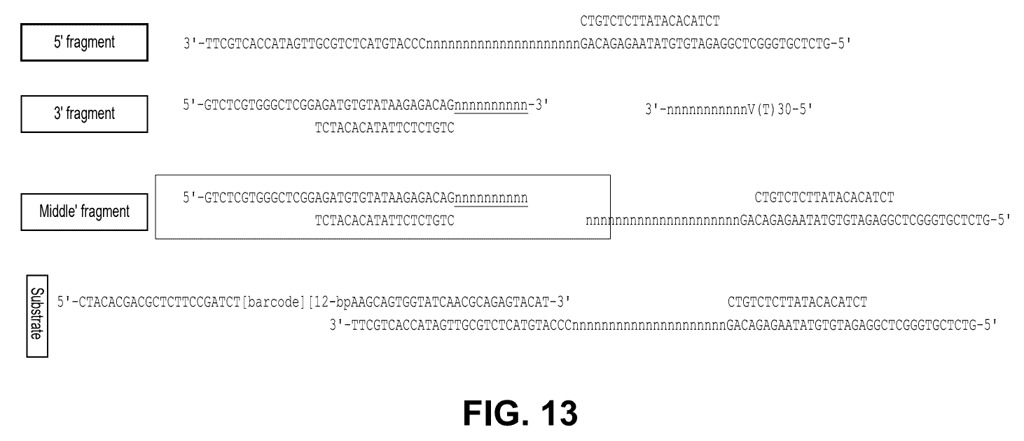

FIG. 13 is a schematic showing remnants of the three different cDNA:RNA fragments (e.g., a 5′ fragment, a 3′ fragment, and a middle fragment) from FIG. 12 after releasing the target RNA from the cDNA:RNA fragments. FIG. 13 (bottom) shows capture of the 5′ fragment via a capture probe including a capture domain on a substrate.

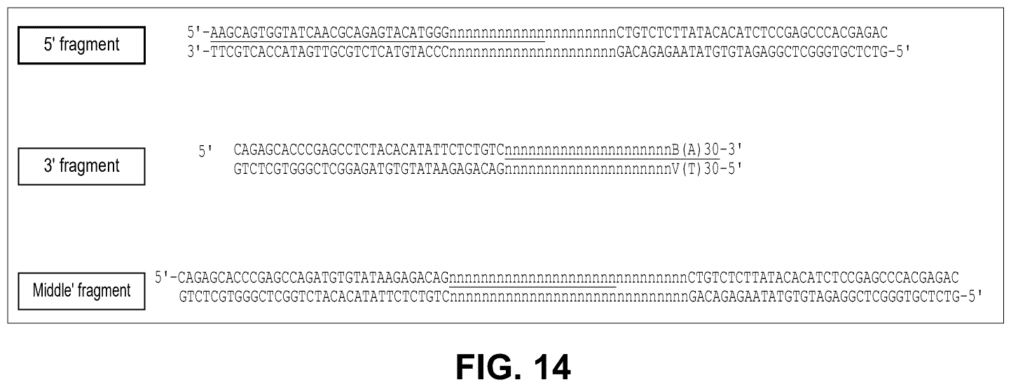

FIG. 14 is a schematic diagram showing the three different cDNA:RNA fragments of FIG. 12 where reverse transcription and/or extension reactions have filled in gaps introduced during tagmentation.

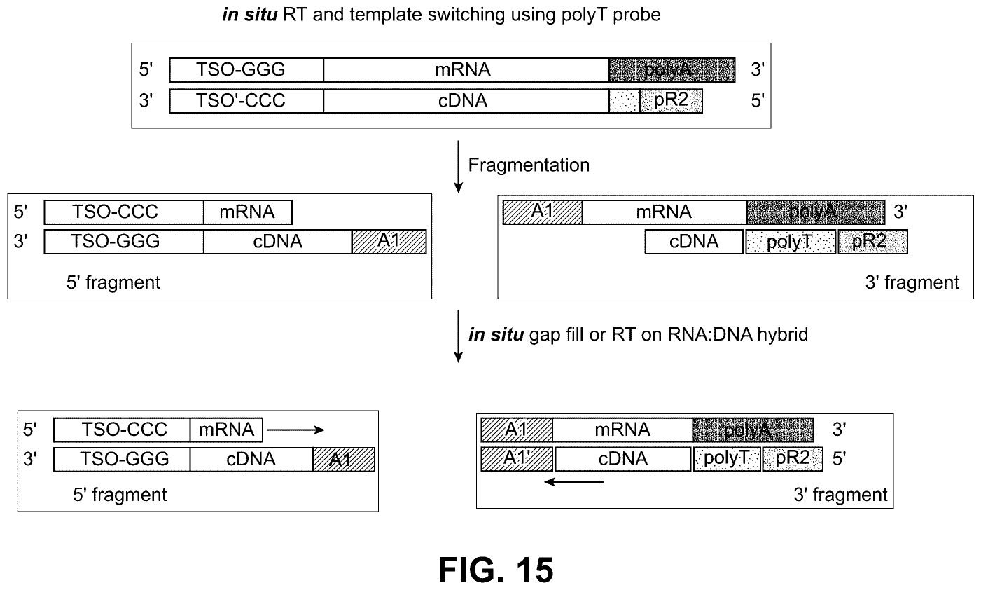

FIG. 15 from top to bottom is a schematic showing an example of an in situ reverse transcribed cDNA product hybridized to a target RNA (top) followed by two different cDNA:RNA fragments (e.g., a 5′ fragment and a 3′ fragment) generated after tagmentation. A middle fragment is also generated, but not shown.

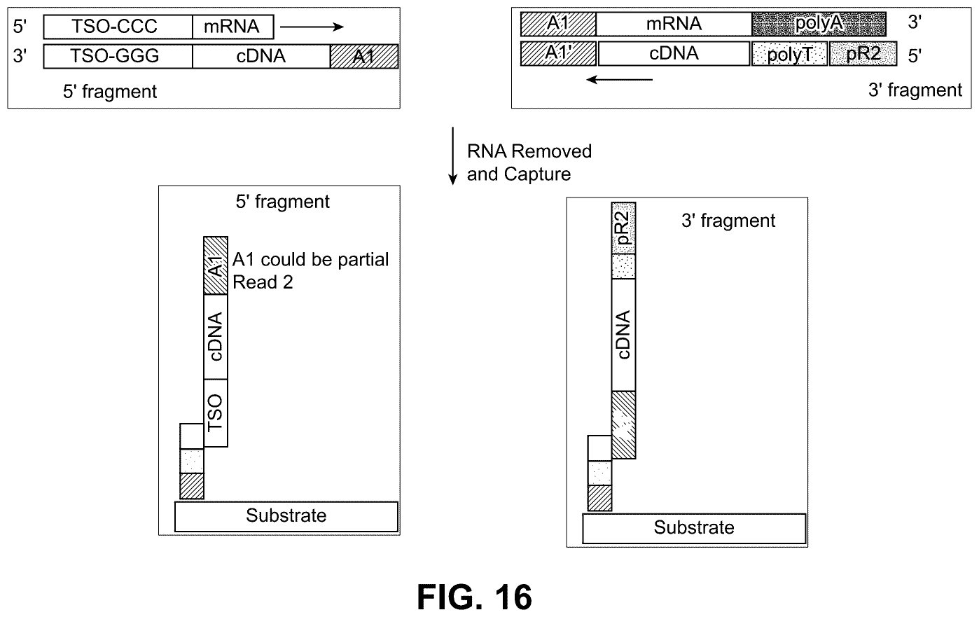

FIG. 16 is a schematic diagram showing release of the 5′ fragment and the 3′ fragment from the target RNA after a gap-fill and/or a reverse transcription reaction and capture of the 5′ and 3′ fragments on a spatial array with capture probes.

### DETAILED DESCRIPTION

### A. Spatial Analysis Methods

Spatial analysis methodologies described herein can provide a vast amount of analyte and/or expression data for a variety of analytes within a biological sample at high spatial resolution, while retaining native spatial context. Spatial analysis methods can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 11,447,807, 11,352,667, 11,168,350, 11,104,936, 11,008,608, 10,995,361, 10,913,975, 10,774,374, 10,724,078, 10,640,816, 10,494,662, 10,480,022, 10,364,457, 10,317,321, 10,059,990, 10,041,949, 10,030,261, 10,002,316, 9,879,313, 9,783,841, 9,727,810, 9,593,365, 8,951,726, 8,604,182, and 7,709,198; U.S. Patent Application Publication Nos. 2020/0239946, 2020/0080136, 2020/0277663, 2019/0330617, 2020/0256867, 2020/0224244, 2019/0085383, and 2013/0171621; PCT Publication Nos. WO2018/091676, WO2020/176788,

WO2017/144338, and WO2016/057552; Non-patent literature references Rodriques et al., Science 363 (6434):1463-1467, 2019; Lee et al., Nat. Protoc. 10(3):442-458, 2015; Trejo et al., PLOS ONE 14 (2):e0212031, 2019; Chen et al., Science 348 (6233):aaa6090, 2015; Gao et al., BMC Biol. 15:50, 2017; and Gupta et al., Nature Biotechnol. 36:1197-1202, 2018; and the Visium Spatial Gene Expression Reagent Kits User Guide (e.g., Rev F, dated January 2022) and/or the Visium Spatial Gene Expression Reagent Kits—Tissue Optimization User Guide (e.g., Rev E, dated February 2022), both of which are available at the 10× Genomics Support Documentation website, and can be used herein in any combination, and each of which is incorporated herein by reference in its entirety. Further non-limiting aspects of spatial analysis methodologies and compositions are described herein.

Some general terminology that may be used in this disclosure can be found in Section (I)(b) of PCT Publication No. WO2020/176788 and/or U.S. Patent Application Publication No. 2020/0277663, which is herein incorporated by reference. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of PCT Publication No. WO2020/176788 and/or U.S. Patent Application Publication No. 2020/0277663, which is herein incorporated by reference. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, the biological sample is a tissue sample. In some embodiments, the biological sample (e.g., tissue sample) is a tissue microarray (TMA). A tissue microarray contains multiple representative tissue samples-which can be from different tissues or organisms-assembled on a single histologic slide. The TMA can therefore allow for high throughput analysis of multiple specimens at the same time. Tissue microarrays may be paraffin blocks produced by extracting cylindrical tissue cores from different paraffin donor blocks and re-embedding these tissue cores into a single recipient (microarray) block at defined array coordinates.

The biological sample as used herein can be any suitable biological sample described herein or known in the art. In some embodiments, the biological sample is a tissue sample. In some embodiments, the tissue sample is a solid tissue sample. In some embodiments, the biological sample is a tissue section (e.g., a fixed tissue section). In some embodiments, the tissue is flash-frozen and sectioned. Any suitable method described herein or known in the art can be used to flash-freeze and section the tissue sample. In some embodiments, the biological sample, e.g., the tissue, is flash-frozen using liquid nitrogen before sectioning. In some embodiments, the biological sample, e.g., a tissue sample, is flash-frozen using nitrogen (e.g., liquid nitrogen), isopentane, or hexane.

In some embodiments, the biological sample, e.g., the tissue, is embedded in a matrix e.g., optimal cutting temperature (OCT) compound to facilitate sectioning. OCT compound is a formulation of clear, water-soluble glycols and resins, providing a solid matrix to encapsulate biological (e.g., tissue) specimens. In some embodiments, the sectioning is performed by cryosectioning, for example using a microtome. In some embodiments, the methods further comprise a thawing step, after the cryosectioning.

The biological sample can be from a mammal. In some instances, the biological sample is from a human, mouse, or rat. In addition to the subjects described above, the biological sample can be obtained from non-mammalian organisms (e.g., a plant, an insect, an arachnid, a nematode (e.g., Caenorhabditis elegans), a fungus, an amphibian, or a fish (e.g., zebrafish)). A biological sample can be obtained from a prokaryote such as a bacterium, e.g., Escherichia coli, Staphylococci or Mycoplasma pneumoniae; an archacon; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

In some embodiments, the biological sample, e.g., the tissue sample, is fixed in a fixative including alcohol, for example, methanol. In some embodiments, instead of methanol, acetone or an acetone-methanol mixture can be used. In some embodiments, the fixation is performed after sectioning. In some instances, when the biological sample is fixed using a fixative including an alcohol (e.g., methanol or acetone-methanol mixture), the biological sample is not decrosslinked afterward. In some preferred embodiments, the biological sample is fixed using a fixative including an alcohol (e.g., methanol or an acetone-methanol mixture) after freezing and/or sectioning. In some instances, the biological sample is flash-frozen, and then the biological sample is sectioned and fixed (e.g., using methanol, acetone, or an acetone-methanol mixture). In some instances when methanol, acetone, or an acetone-methanol mixture is used to fix the biological sample, the sample is not decrosslinked at a later step. In instances when the biological sample is frozen (e.g., flash frozen using liquid nitrogen and embedded in OCT) followed by sectioning and alcohol (e.g., methanol, acetone-methanol) fixation or acetone fixation, the biological sample is referred to as “fresh frozen”. In some embodiments, fixation of the biological sample, e.g., using acetone and/or alcohol (e.g., methanol, acetone-methanol), is performed while the sample is mounted on a substrate (e.g., glass slide, such as a positively charged glass slide).

In some embodiments, a substrate of the present technology includes a surface comprising one or more spatially barcoded capture probes, wherein the spatial barcodes are present at known spatial locations on the substrate. In some embodiments, a substrate of the present technology includes a surface comprising one or more spatially barcoded capture probes that are arranged in an ordered manner, such as a grid. In some embodiments, a substrate of the present technology includes a surface comprising one or more spatially barcoded capture probes, wherein the spatially barcoded capture probes are provided in a known but non-ordered manner, such as a random or irregular manner.

In some embodiments, a substrate of the present technology comprises an array (such as an ordered or non-ordered array). In some embodiments, a substrate of the present technology comprises an array of spatially barcoded capture probes present on the substrate surface in an ordered manner, such as a grid. In some embodiments, a substrate of the present technology includes a surface comprising an array of spatially barcoded capture probes present on the substrate surface in a non-ordered manner, such as a random or irregular manner.

In some embodiments, the biological sample, e.g., the tissue sample, is fixed e.g., immediately after being harvested from a subject. In such embodiments, the fixative is preferably an aldehyde fixative, such as paraformaldehyde (PFA) or formalin. In some embodiments, the fixative induces crosslinks within the biological sample. In some embodiments, after fixing, e.g., by formalin or PFA, the biological sample is dehydrated via sucrose gradient. In some instances, the fixed biological sample is treated with a sucrose gradient and then embedded in a matrix, e.g., OCT compound. In some instances, the fixed biological sample is not treated with a sucrose gradient, but rather is embedded in a matrix, e.g., OCT compound after fixation. In some embodiments when a fixed frozen tissue sample is treated with a sucrose gradient, the sample can be rehydrated using an ethanol gradient. In some embodiments, the PFA or formalin fixed biological sample, which can be optionally dehydrated via sucrose gradient and/or embedded in OCT compound, is then frozen, e.g., for storage or shipment. In such instances, the biological sample is referred to as “fixed frozen”. In preferred embodiments, a fixed frozen biological sample is not treated with methanol. In preferred embodiments, a fixed frozen biological sample is not paraffin embedded. Thus, in preferred embodiments, a fixed frozen biological sample is not deparaffinized. In some embodiments, a fixed frozen biological sample is rehydrated using an ethanol gradient.

In some instances, the biological sample (e.g., a fixed frozen tissue sample) is treated with a citrate buffer. Citrate buffer can be used to decrosslink antigens and fixation medium for antigen retrieval in the biological sample. Thus, any suitable decrosslinking agent can be used in addition, or alternatively, to citrate buffer. In some embodiments, for example, the biological sample (e.g., a fixed frozen tissue sample) is decrosslinked using TE buffer.

In any of the foregoing, the biological sample can further be stained, imaged, and/or destained. For example, in some embodiments, a fresh frozen tissue sample or fixed frozen tissue sample is stained (e.g., via cosin and/or hematoxylin), imaged, destained (e.g., via HCl), or a combination thereof. In some embodiments, when a fresh frozen tissue sample is fixed in methanol, the sample is treated with isopropanol prior to being stained (e.g., via cosin and/or hematoxylin), imaged, destained (e.g., via HCl), or a combination thereof. In some embodiments when a fixed frozen tissue sample is treated with a sucrose gradient, the sample can be rehydrated using an ethanol gradient before being stained, (e.g., via cosin and/or hematoxylin), imaged, destained (e.g., via HCl), decrosslinked (e.g., via TE buffer or citrate buffer), or a combination thereof. In some embodiments, the biological sample can undergo further fixation (e.g., while mounted on a substrate), stained, imaged, and/or destained. For example, a fixed frozen biological sample may be subject to an additional fixing step (e.g., using PFA) before optional ethanol rehydration, staining, imaging, and/or destaining.

In any of the foregoing, the biological sample can be fixed using PAXgene. For example, the biological sample can be fixed using PAXgene in addition, or alternatively to, a fixative disclosed herein or known in the art (e.g., alcohol, acetone, acetone-alcohol, formalin, paraformaldehyde). PAXgene is a non-cross-linking mixture of different alcohols, an acid, and a soluble organic compound that preserves morphology and biomolecules. PAXgene provides a two-reagent fixative system in which tissue is firstly fixed in a solution containing methanol and acetic acid, then stabilized in a solution containing ethanol. See, Ergin B. et al., J Proteome Res. 2010 Oct. 1; 9(10):5188-96; Kap M. et al., PLOS One.; 6(11):e27704 (2011); and Mathieson W. et al., Am J Clin Pathol.; 146(1):25-40 (2016), each of which is hereby incorporated by reference in its entirety, for a description and evaluation of PAXgene for tissue fixation. Thus, in some embodiments, when the biological sample, e.g., the tissue sample, is fixed in a fixative including alcohol, the fixative is PAXgene. In some embodiments, a fresh frozen tissue sample is fixed with PAXgene. In some embodiments, a fixed frozen tissue sample is fixed with PAXgene.

In some embodiments, the biological sample, e.g., the tissue sample, is fixed, for example in methanol, acetone, acetone-methanol, PFA, PAXgene, or is formalin-fixed and paraffin-embedded (FFPE). In some embodiments, the biological sample comprises intact cells. In some embodiments, the biological sample is a cell pellet, e.g., a fixed cell pellet, e.g., an FFPE cell pellet. FFPE samples are used in some instances in the RNA-templated ligation (RTL) methods disclosed herein. A limitation of direct RNA capture for fixed samples is that the RNA integrity of fixed (e.g., FFPE) samples can be lower than of a fresh sample, thereby capturing RNA directly from fixed samples, e.g., by capture of a common sequence such as a poly(A) tail of an mRNA molecule, can be more difficult. By utilizing RTL probes that hybridize to RNA target sequences in the transcriptome, RNA analytes can be captured without requiring that both a poly(A) tail and target sequences remain intact. Accordingly, RTL probes can be utilized to beneficially improve capture and spatial analysis of fixed samples. The biological sample, e.g., tissue sample, can be stained, and imaged prior, during, and/or after each step of the methods described herein. Any of the methods described herein or known in the art can be used to stain and/or image the biological sample. In some embodiments, the imaging occurs prior to destaining the sample. In some embodiments, the biological sample is stained using an H&E staining method. In some embodiments, the tissue sample is stained and imaged for about 10 minutes to about 2 hours (or any of the subranges of this range described herein). Additional time may be needed for staining and imaging of different types of biological samples.

The tissue sample can be obtained from any suitable location in a tissue or organ of a subject, e.g., a human subject. In some instances, the sample is a mouse sample. In some instances, the sample is a human sample. In some embodiments, the sample can be derived from skin, brain, breast, lung, liver, kidney, prostate, tonsil, thymus, testes, bone, lymph node, ovary, eye, heart, or spleen. In some instances, the sample is a human or mouse breast tissue sample. In some instances, the sample is a human or mouse brain tissue sample. In some instances, the sample is a human or mouse lung tissue sample. In some instances, the sample is a human or mouse tonsil tissue sample. In some instances, the sample is a human or mouse liver tissue sample. In some instances, the sample is a human or mouse bone, skin, kidney, thymus, testes, or prostate tissue sample. In some embodiments, the tissue sample is derived from normal or diseased tissue. In some embodiments, the sample is an embryo sample. The embryo sample can be a non-human embryo sample. In some instances, the sample is a mouse embryo sample.

Biological samples are also described in Section (I)(d) of PCT Publication No. WO2020/176788 and/or U.S. Patent Application Publication No. 2020/0277663, which is herein incorporated by reference.

The following embodiments can be used with any of the methods described herein. In some embodiments, the biological sample (e.g., a fixed and/or stained biological sample) is imaged. In some embodiments, the biological sample is visualized or imaged using bright field microscopy. In some embodiments, the biological sample is visualized or imaged using fluorescence microscopy. The biological sample can be visualized or imaged using additional methods of visualization and imaging known in the art. Non-limiting examples of visualization and imaging include expansion microscopy, bright field microscopy, dark field microscopy, phase contrast microscopy, electron microscopy, fluorescence microscopy, reflection microscopy, interference microscopy and confocal microscopy. In some embodiments, the sample is stained and imaged prior to adding reagents for analyzing captured analytes, as disclosed herein, to the biological sample.

In some embodiments, the methods include staining the biological sample. In some embodiments, the staining includes the use of hematoxylin and/or cosin. Non-limiting examples of stains include histological stains (e.g., hematoxylin and/or cosin) and immunological stains (e.g., fluorescent stains). In some embodiments, a biological sample can be stained using any number of biological stains, including but not limited to, acridine orange, Bismarck brown, carmine, coomassie blue, cresyl violet, DAPI (4′,6-diamidino-2-phenylindole), cosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the biological sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the staining includes the use of a detectable label, such as a radioisotope, a fluorophore, a chemiluminescent compound, a bioluminescent compound, or a combination thereof.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of PCT Publication No. WO2020/176788 and/or U.S. Patent Application Publication No. 2020/0277663, which is herein incorporated by reference. Briefly, any of the methods described herein includes permeabilizing the biological sample. For example, the biological sample can be permeabilized to facilitate transfer of extension products to the capture probes on the array. In some embodiments, the permeabilizing includes the use of an organic solvent (e.g., acetone, ethanol, or methanol), a detergent (e.g., saponin, Triton X-100™, Tween-20™, or sodium dodecyl sulfate (SDS)), an enzyme (e.g., an endopeptidase, an exopeptidase, or a protease), or a combination thereof. In some embodiments, the permeabilizing includes the use of an endopeptidase, a protease, SDS, polyethylene glycol tert-octylphenyl ether, polysorbate 80, polysorbate 20, N-lauroylsarcosine sodium salt solution, saponin, Triton X-100™, Tween-20™, or a combination thereof. In some embodiments, the endopeptidase is pepsin. In some embodiments, the endopeptidase is Proteinase K. Additional methods for sample permeabilization are described, for example, in Jamur et al., Method Mol. Biol. 588:63-66, 2010, which is herein incorporated by reference.

Array-based spatial analysis methods can involve the transfer of one or more analytes or derivatives thereof from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI) and a capture domain). In some instances, the capture probe includes a homopolymer sequence, such as a poly(T) sequence. In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of PCT Publication No. WO2020/176788 and/or U.S. Patent Application Publication No. 2020/0277663, which is herein incorporated by reference. Generation of capture probes can be achieved by any appropriate method, including those described in Section (II)(d)(ii) of PCT Publication No. WO2020/176788 and/or U.S. Patent Application Publication No. 2020/0277663, which is herein incorporated by reference.

In some instances, a capture probe and a nucleic acid analyte interaction (or any other nucleic acid to nucleic acid interaction) occurs because the sequences of the two nucleic acids are substantially complementary to one another. By “substantial,” “substantially,” and the like, two nucleic acid sequences can be complementary when at least 60% of the nucleotide residues of one nucleic acid sequence are complementary to nucleotide residues of the other nucleic acid sequence. The complementary residues within a particular complementary nucleic acid sequence need not always be contiguous with each other, but can be interrupted by one or more non-complementary residues within the complementary nucleic acid sequence. In some embodiments, at least 60%, but less than 100%, of the residues of one of the two complementary nucleic acid sequences are complementary to residues of the other nucleic acid sequence. In some embodiments, at least 70%, 80%, 90%, 95%, or 99% of the residues of one nucleic acid sequence are complementary to residues of the other nucleic acid sequence. Sequences are said to be “substantially complementary” when at least 60% (e.g., at least 70%, at least 80%, or at least 90%) of the residues of one nucleic acid sequence are complementary to residues of the other nucleic acid sequence. In some embodiments, the biological sample is mounted on a first substrate and the substrate comprising the array of capture probes is a second substrate. In this configuration, one or more analytes or analyte derivatives (e.g., intermediate agents; e.g., ligation products) are then released from the biological sample and migrate to the second substrate comprising an array of capture probes. In some embodiments, the release and migration of the analytes or analyte derivatives to the second substrate comprising the array of capture probes occurs in a manner that preserves the original spatial context of the analytes in the biological sample. This method can be referred to as a sandwiching process, which is described, e.g., in U.S. Patent Application Pub. No. 2021/0189475 and PCT Pub. Nos. WO 2021/252747 A1, WO 2022/061152 A2, and WO 2022/140028 A1, each of which is herein incorporated by reference.

FIG. 1A shows an exemplary sandwiching process 100 where a first substrate (e.g., slide 103), including a biological sample 102, and a second substrate (e.g., array slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., array slide 104) is in an inferior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution which permeabilizes and/or digests the biological sample 102. In some embodiments wherein the biological sample 102 has been pre-permeabilized, the reagent medium is not a permeabilization solution. Herein, the reagent medium may also comprise one or more of a monovalent salt, a divalent salt, ethylene carbonate, and/or glycerol. In some embodiments, analytes (e.g., mRNA transcripts) and/or analyte derivatives (e.g., intermediate agents; e.g., ligation products) of the biological sample 102 may release from the biological sample, and actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788 and U.S. Patent Application Pub. No. 2021/0189475, each of which is hereby incorporated by reference in its entirety.

As further shown, one or more spacers 110 may be positioned between the first substrate (e.g., slide 103) and the second substrate (e.g., array slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns (μm) and about 1 mm (e.g., between about 2 μm and about 800 μm, between about 2 μm and about 700 μm, between about 2 μm and about 600 μm, between about 2 μm and about 500 μm, between about 2 μm and about 400 μm, between about 2 μm and about 300 μm, between about 2 μm and about 200 μm, between about 2 μm and about 100 μm, between about 2 μm and about 25 μm, or between about 2 μm and about 10 μm), measured in a direction orthogonal to the surface of first substrate that supports the biological sample. In some instances, the separation distance is about 2, 3, 4, 5, 6, 7, 8, 9, 10, 11, 12, 13, 14, 15, 16, 17, 18, 19, 20, 21, 22, 23, 24, or 25 μm. In some embodiments, the separation distance is less than 50 μm. In some embodiments, the separation distance is less than 25 μm. In some embodiments, the separation distance is less than 20 μm. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate (e.g., slide 103), and the second substrate (e.g., slide 104) may reduce or prevent undesirable movement (e.g., convective movement) of transcripts and/or molecules during the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., U.S. Patent Application Pub. No. 2021/0189475 and PCT Publ. No. WO 2022/061152 A2, each of which is incorporated by reference in its entirety.

In some embodiments of a sample holder, the sample holder can include a first member including a first retaining mechanism configured to retain a first substrate comprising a biological sample. The first retaining mechanism can be configured to retain the first substrate disposed in a first plane. The sample holder can further include a second member including a second retaining mechanism configured to retain a second substrate disposed in a second plane. The sample holder can further include an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane of the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes of the array and may be processed for spatial analysis.

In some embodiments, during the permeabilization step, the image capture device 220 may capture images of the overlap area between the biological sample and the capture probes on the array 106. If more than one first substrates 206 and/or second substrates 212 are present within the sample handling apparatus 200, the image capture device 220 may be configured to capture one or more images of one or more overlap areas.

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some exemplary implementations.

FIG. 3A depicts the first substrate (e.g., slide 303 including a biological sample 302) angled over (superior to) the second substrate (e.g., slide 304). As shown, reagent medium (e.g., permeabilization solution) 305 is located on the spacer 310 toward the right-hand side of the side view in FIG. 3A. While FIG. 3A depicts the reagent medium on the right-hand side of side view, it should be understood that such depiction is not meant to be limiting as to the location of the reagent medium on the spacer.

FIG. 3B shows that as the first substrate lowers and/or as the second substrate rises, the dropped side of the first substrate (e.g., a side of the slide 303 angled toward the slide 304) may contact the reagent medium 305. The dropped side of the slide 303 may urge the reagent medium 305 toward the opposite direction (e.g., towards an opposite side of the spacer 310, towards an opposite side of the slide 303 relative to the dropped side). For example, in the side view of FIG. 3B the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 form the sides of chamber 350 which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the substrates to facilitate transfer of target analytes with spatial information. Additionally, air bubbles present between the substrates may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104). In some aspects, it may be possible to reduce or eliminate bubble formation between the substrates using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

FIG. 4A is a side view of the angled closure workflow 400 in accordance with some exemplary implementations. FIG. 4B is a top view of the angled closure workflow 400 in accordance with some exemplary implementations. As shown at step 405, reagent medium 401 is positioned to the side of the substrate 402.

At step 410, the dropped side of the angled substrate 406 contacts the reagent medium 401 first. The contact of the substrate 406 with the reagent medium 401 may form a linear or low curvature flow front that fills the gap between the two substrates 406 and 402 uniformly with the slides closed.

At step 415, the substrate 406 is further lowered toward the substrate 402 (or the substrate 402 is raised up toward the substrate 406) and the dropped side of the substrate 406 may contact and urge the reagent medium toward the side opposite the dropped side, thereby creating a linear or low curvature flow front that may prevent or reduce bubble trapping between the substrates.

At step 420, the reagent medium 401 fills the gap between the substrate 406 and the substrate 402. The linear flow front of the liquid reagent may be formed by squeezing the reagent medium 401 volume along the contact side of the substrate 402 and/or the substrate 406. Additionally, capillary flow may also contribute to filling the gap area.

In some embodiments, the reagent medium (e.g., 105 in FIG. 1A) comprises a permeabilization agent. In some embodiments, following initial contact between the biological sample and a permeabilization agent, the permeabilization agent can be removed from contact with the biological sample (e.g., by opening the sample holder). Suitable agents for this purpose include, but are not limited to, organic solvents (e.g., acetone, ethanol, or methanol), cross-linking agents (e.g., paraformaldehyde), detergents (e.g., saponin, Triton X-100™, Tween-20™, SDS), and enzymes (e.g., trypsin or other proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and SDS. More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, elastase, and proteinase K. In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of SDS or a sodium salt thereof, proteinase K, pepsin, N-lauroylsarcosine, and RNase.

In some embodiments, the reagent medium comprises polyethylene glycol (PEG). In some embodiments, the PEG molecular weight is from about 2K to about 16K. In some embodiments, the PEG is about 2K, about 3K, about 4K, about 5K, about 6K, about 7K, about 8K, about 9K, about 10K, about 11K, about 12K, about 13K, about 14K, about 15K, or about 16K. In some embodiments, the PEG is present at a concentration from about 2% to about 25%, from about 4% to about 23%, from about 6% to about 21%, or from about 8% to about 20% (v/v).

In certain embodiments, a dried permeabilization reagent is applied or formed as a layer on the first substrate, the second substrate, or both prior to contacting the biological sample with the array. For example, a permeabilization reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of PCT Publication No. WO2020/176788 and/or U.S. Patent Application Publication No. 2020/0277663 regarding extended capture probes, which is herein incorporated by reference). In some cases, capture probes may be configured to form ligation products with a template (e.g., a DNA or RNA template, such as an analyte or an intermediate agent, or portion thereof), thereby creating ligation products that serve as proxies for the template.

As used herein, an “extended capture probe” refers to a capture probe having additional nucleotides added to a terminus (e.g., a 3′ or 5′ end) of the capture probe, thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using a reverse transcriptase. In some embodiments, the capture probe is extended using one or more DNA polymerases. In some embodiments, the extended capture probes include the sequence of the capture domain, the sequence of the spatial barcode of the capture probe, and the complementary sequence of the template used for extension of the capture probe.

In some embodiments, extended capture probes are amplified (e.g., in bulk solution or on the array) to yield quantities that are sufficient for downstream analysis, e.g., sequencing. In some embodiments, extended capture probes (e.g., DNA molecules) can act as templates for an amplification reaction (e.g., a polymerase chain reaction).

Additional variants of spatial analysis methods, including in some embodiments, an imaging step, are described in Section (II)(a) of PCT Publication No. WO2020/176788 and/or U.S. Patent Application Publication No. 2020/0277663, which is herein incorporated by reference. Analysis of captured analytes (and/or intermediate agents or portions thereof), for example, including sample removal, extension of capture probes using the captured analyte as a template, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of PCT Publication No. WO2020/176788 and/or U.S. Patent Application Publication No. 2020/0277663, which is herein incorporated by reference. Some quality control measures are described in Section (II)(h) of PCT Publication No. WO2020/176788 and/or U.S. Patent Application Publication No. 2020/0277663, which is herein incorporated by reference.

Spatial information can provide information of medical importance. For example, the methods described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication Nos. 2021/0140982, 2021/0198741, and 2021/0199660, each of which is herein incorporated by reference in its entirety.

Spatial information can provide information of biological importance. For example, the methods described herein can allow for: identification of transcriptome and/or proteome expression profiles (e.g., in healthy and/or diseased tissue); identification of multiple analyte types in close proximity (e.g., nearest neighbor or proximity based analysis); determination of up-regulated and/or down-regulated genes and/or proteins in diseased tissue; characterization of tumor microenvironments; characterization of tumor immune responses; characterization of cells types and their co-localization in healthy and diseased tissue; and identification of genetic variants within tissues (e.g., based on gene and/or protein expression profiles associated with specific disease or disorder biomarkers).

For spatial array-based methods, a substrate may function as a support for direct or indirect attachment of capture probes to features of the array. A “feature” is an entity that acts as a support or repository for various molecular entities used in spatial analysis. In some embodiments, some or all of the features in an array are functionalized for analyte capture.

Exemplary substrates are described in Section (II) (c) of PCT Publication No. WO2020/176788 and/or U.S. Patent Application Publication No. 2020/0277663, which is herein incorporated by reference. Exemplary features and geometric attributes of an array can be found in Sections (II)(d)(i), (II)(d)(iii), and (II)(d)(iv) of PCT Publication No. WO2020/176788 and/or U.S. Patent Application Publication No. 2020/0277663, which is herein incorporated by reference.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads or wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(c) of PCT Publication No. WO2020/176788 and/or U.S. Patent Application Publication No. 2020/0277663, which is herein incorporated by reference.

FIG. 5 is a schematic diagram showing an exemplary capture probe, as described herein. As shown, the capture probe 502 is optionally coupled to a feature 501 by a cleavage domain 503, such as a disulfide linker. The capture probe can include a functional sequence 504 that is useful for subsequent processing. The functional sequence 504 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 505. The capture probe can also include a unique molecular identifier (UMI) sequence 506. While FIG. 5 shows the spatial barcode 505 as being located upstream (5′) of UMI sequence 506, it is to be understood that capture probes wherein UMI sequence 506 is located upstream (5′) of the spatial barcode 505 is also suitable for use in any of the methods described herein. The capture probe can also include a capture domain 507 to facilitate capture of a target analyte. The capture domain can have a sequence complementary to a sequence of a nucleic acid analyte. The capture domain can have a sequence complementary to a connected probe described herein. The capture domain can have a sequence complementary to an analyte capture sequence present in an analyte capture agent. The capture domain can have a sequence complementary to a splint oligonucleotide. A splint oligonucleotide, in addition to having a sequence complementary to a capture domain of a capture probe, can have a sequence complementary to a sequence of a nucleic acid analyte, a portion of a connected probe described herein, a capture handle sequence described herein, and/or a methylated adaptor described herein.

FIG. 6 is a schematic illustrating a cleavable capture probe, wherein the cleaved capture probe can enter into a non-permeabilized cell and bind to analytes within the cell. The capture probe 601 can contain a cleavage domain 602, a cell penetrating peptide 603, a reporter molecule 604, and a disulfide bond (—S—S—). 605 represents all other parts of a capture probe, for example, a spatial barcode and a capture domain.

FIG. 7 is a schematic diagram of an exemplary multiplexed spatially-barcoded feature. In FIG. 7, the feature 701 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may include four different types of spatially-barcoded capture probes, each type of spatially-barcoded capture probe possessing the spatial barcode 702. One type of capture probe associated with the feature can include the spatial barcode 702 in combination with a poly(T) capture domain 703, designed to capture mRNA target analytes. A second type of capture probe associated with the feature can include the spatial barcode 702 in combination with a random N-mer capture domain 704 for gDNA analysis. A third type of capture probe associated with the feature can include the spatial barcode 702 in combination with a capture domain complementary to the analyte capture agent of interest 705. A fourth type of capture probe associated with the feature can include the spatial barcode 702 in combination with a capture probe that can specifically bind a nucleic acid molecule 706 that can function in a CRISPR assay (e.g., CRISPR/Cas9). While only four different capture probe-barcoded constructs are shown in FIG. 7, capture-probe barcoded constructs can be tailored for analyses of any given analyte associated with a nucleic acid and capable of binding with such a construct. For example, the schemes shown in FIG. 7 can also be used for concurrent analysis of other analytes disclosed herein, including, but not limited to: (a) mRNA, a lineage tracing construct, cell surface or intracellular proteins and/or metabolites, and gDNA; (b) mRNA, accessible chromatin (e.g., ATAC-seq, DNase-seq, and/or MNase-seq), cell surface or intracellular proteins and/or metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature) change, or any other known perturbation agents.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 505 and functional sequence 504 are common to all of the probes attached to a given feature. In some embodiments, the UMI sequence 506 of a capture probe attached to a given feature is different from the UMI sequence of a different capture probe attached to the given feature.

FIG. 8 depicts an exemplary arrangement of barcoded features within an array. From left to right, FIG. 8 shows (left) a slide including six spatially-barcoded arrays, (center) an enlarged schematic of one of the six spatially-barcoded arrays, showing a grid of barcoded features in relation to a biological sample, and (right) an enlarged schematic of one section of an array, showing the specific identification of multiple features within the array (e.g., labelled as ID578, ID579, ID580, etc.).

In some embodiments, more than one analyte type (e.g., nucleic acids and proteins) from a biological sample can be detected (e.g., simultaneously or sequentially) using any appropriate multiplexing technique, such as those described in Section (IV) of PCT Publication No. WO2020/176788 and/or U.S. Patent Application Publication No. 2020/0277663, which is herein incorporated by reference.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of PCT Publication No. WO2020/176788 and/or U.S. Patent Application Publication No. 2020/0277663, which is herein incorporated by reference.

In some cases, spatial analysis can be performed by detecting multiple oligonucleotides that hybridize to an analyte. In some instances, for example, spatial analysis can be performed using RNA-templated ligation (RTL). Methods of RTL have been described previously. Sec, e.g., Credle et al., Nucleic Acids Res. 2017 Aug. 21; 45(14):e128, which is herein incorporated by reference in its entirety. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture probe binding domain (e.g., a poly(A) sequence or a non-homopolymeric sequence). After hybridization to the analyte, a ligase (e.g., a T4 RNA ligase (Rnl2), a PBCV-1 DNA Ligase or Chlorella virus DNA Ligase, a single-stranded DNA ligase, or a T4 DNA ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNase H). In some instances, the ligation product is removed using heat. In some instances, the ligation product is removed using KOH. The released ligation product can then be captured by capture probes (e.g., instead of direct capture of an analyte) on an array, optionally amplified, and sequenced, thus determining the location, and optionally, the abundance of the analyte in the biological sample.

In some instances, one or both of the oligonucleotides may hybridize to genomic DNA (gDNA), which can lead to false positive sequencing data from ligation events on gDNA (off target) in addition to the desired (on target) ligation events on target nucleic acids (e.g., mRNA). Thus, in some embodiments, the disclosed methods can include contacting the biological sample with a deoxyribonuclease (DNase). The DNase can be an endonuclease or exonuclease. In some embodiments, the DNase digests single-stranded and/or double-stranded DNA. Suitable DNases include, without limitation, a DNase I and a DNase II. Use of a DNase as described can mitigate false positive sequencing data from off target gDNA ligation events.

A non-limiting example of templated ligation methods disclosed herein is depicted in FIG. 9A. After a biological sample is contacted with a substrate including a plurality of capture probes and contacted with (a) a first probe 901 having a target-hybridization sequence 903 and a primer sequence 902 and (b) a second probe 904 having a target-hybridization sequence 905 and a capture domain (e.g., a poly(A) sequence) 906, the first probe 901 and the second probe 904 hybridize 910 to an analyte 907. A ligase 921 ligates 920 the first probe 901 to the second probe 904, thereby generating a ligation product 922. The ligation product 922 is then released 930 from the analyte 931 by digesting the analyte 907 using an endoribonuclease 932. The sample is permeabilized 940 and the ligation product 941 is able to hybridize to a capture probe on the substrate. Methods and compositions for spatial detection using templated ligation have been described in PCT Publication. No. WO 2021/133849 A1, U.S. Pat. Nos. 11,332,790 and 11,505,828, each of which is incorporated by reference in its entirety.

In some embodiments, as shown in FIG. 9B, the ligation product 9001 includes a capture probe capture domain 9002, which can bind to a capture probe 9003 (e.g., a capture probe immobilized, directly or indirectly, on a substrate 9004). In some embodiments, methods provided herein include contacting 9005 a biological sample with a substrate 9004, wherein the capture probe 9003 is affixed to the substrate (e.g., immobilized to the substrate, directly or indirectly). In some embodiments, the capture probe capture domain 9002 of the ligated product 9001 specifically binds to the capture domain 9006. The capture probe can also include a unique molecular identifier (UMI) 9007, a spatial barcode 9008, a functional sequence 9009, and a cleavage domain 9010.

In some embodiments, methods provided herein include permeabilization of the biological sample such that the capture probe can more easily capture the ligation products (i.e., compared to no permeabilization). In some embodiments, polymerization (e.g., reverse transcription (RT)) reagents can be added to permeabilized biological samples. Incubation with the polymerization reagents can be used to extend the capture probes 9011 to produce spatially-barcoded full-length cDNA 9012 and 9013 from the captured ligation products (e.g., ligation products). The ligation products can be extended using the capture probe as a template to include a complement of the capture probe, thereby generating extended ligation products.

In some embodiments, the extended ligation products can be denatured 9014, released from the capture probe, and transferred (e.g., to a clean tube) for amplification and/or library construction. The spatially-barcoded ligation products can be amplified 9015 via PCR prior to library construction. P5 9016, i5 9017, i7 9018, and P7 9019 sequences can be used as sample indexes. The amplicons can then be sequenced using paired-end sequencing using TruSeq Read 1 and TruSeq Read 2 as sequencing primer sites.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of PCT Publication No. WO2020/176788 and/or Section (II)(b)(viii) U.S. Patent Application Publication No. 2020/0277663, which is herein incorporated by reference.

FIG. 10 is a schematic diagram of an exemplary analyte capture agent 1002 comprised of an analyte binding moiety 1004 and an analyte-binding moiety barcode domain 1008. The exemplary analyte binding moiety 1004 is capable of binding to an analyte 1006 and the analyte capture agent 1002 is capable of interacting with a spatially-barcoded capture probe. The analyte binding moiety 1004 can bind to the analyte 1006 with high affinity and/or with high specificity. The analyte capture agent 1002 can include: (i) an analyte binding moiety barcode domain 1008, which serves to identify the analyte binding moiety, and (ii) an analyte capture sequence, which can hybridize to at least a portion or an entirety of a capture domain of a capture probe. The analyte binding moiety 1004 can include a polypeptide and/or an aptamer. The analyte binding moiety 1004 can include an antibody or antibody fragment (e.g., an antigen-binding fragment). FIG. 11 is a schematic diagram depicting an exemplary interaction between a feature-immobilized capture probe 1124 and an analyte capture agent 1126. The feature-immobilized capture probe 1124 can include a spatial barcode 1108 as well as functional sequence 1106 and a UMI 1110, as described elsewhere herein. The capture probe can be affixed 1104 to a feature such as a bead 1102. The capture probe 1124 can also include a capture domain 1112 that is capable of binding to an analyte capture agent 1126. The analyte binding moiety barcode domain of the analyte capture agent 1126 can include functional sequence 1118, analyte binding moiety barcode 1116, and an analyte capture sequence 1114 that is capable of binding (e.g., hybridizing) to the capture domain 1112 of the capture probe 1124. The analyte capture agent 1126 can also include a linker 1120 that allows the analyte binding moiety barcode domain (e.g., including the functional sequence 1118, analyte binding moiety barcode 1116, and analyte capture sequence 1114) to couple to the analyte binding moiety 1122. In some embodiments, the linker 1120 is a cleavable linker. In some embodiments, the cleavable linker is a photo-cleavable linker, a UV-cleavable linker, chemical-cleavable, thermal-cleavable, or an enzyme cleavable linker. In some instances, the cleavable linker is a disulfide linker. A disulfide linker can be cleaved by use of a reducing agent, such as dithiothreitol (DTT), beta-mercaptoethanol (BME), or Tris (2-carboxyethyl) phosphine (TCEP).

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the captured analytes are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that each spatial barcode is uniquely associated with a single feature of the array.

Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location or a fiducial marker) of the array. Accordingly, each feature location has an “address” or location in the coordinate space of the array.

Some exemplary spatial analysis workflows are described in the Exemplary Embodiments section of PCT Publication No. WO2020/176788 and/or U.S. Patent Application Publication No. 2020/0277663, which is herein incorporated by reference. See, for example, the Exemplary embodiment starting with “In some non-limiting examples of the workflows described herein, the sample can be immersed . . . ” of PCT Publication No. WO2020/176788 and/or U.S. Patent Application Publication No. 2020/0277663, which is herein incorporated by reference. See also, e.g., the Visium Spatial Gene Expression Reagent Kits User Guide (e.g., Rev F, dated January 2022) and/or the Visium Spatial Gene Expression Reagent Kits-Tissue Optimization User Guide (e.g., Rev E, dated February 2022), each of which is herein incorporated by reference in its entirety.

In some embodiments, spatial analysis can be performed using dedicated hardware and/or software, such as any of the systems described in Sections (II)(e)(ii) and/or (V) of PCT Publication No. WO2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of PCT Publication No. WO2020/123320, which is herein incorporated by reference.

Suitable systems for performing spatial analysis can include components such as a chamber (e.g., a flow cell or a scalable, fluid-tight chamber) for containing a biological sample. The biological sample can be mounted, for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable, and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Publication No. WO2021/102003 and/or U.S. Patent Application Publication No. 2021/0150707, each of which is incorporated herein by reference in its entirety.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two-dimensional and/or three-dimensional map of the analyte presence and/or level are described in PCT Publication No. WO2020/053655 and spatial analysis methods are generally described in PCT Publication No. WO2021/102039 and/or U.S. Patent Application Publication No. 2021/0155982, each of which is incorporated herein by reference in its entirety.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of PCT Publication Nos. WO2020/123320, WO 2021/102005, and/or U.S. Patent Application Publication No. 2021/0158522, each of which is incorporated herein by reference in its entirety. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### B. Transposase-Mediated Spatial Capture of Target Nucleic Acids or Proxies Thereof

Spatial analysis methods typically rely on detergents and/or enzymes to digest a biological sample (e.g., tissue section) and release transcripts, such as poly(A) mRNA. However, such an assay inherently involves permeabilization optimization for each tissue type, in part because tissue-resident ECM proteins differ across tissue types. As such, optimal permeabilization times tends to differ across sample types. For example, human breast cancer is rich in collagenase and may require a separate collagenase treatment for optimal permeabilization to facilitate release of mRNA molecules as compared to other tissue types. The disclosed methods mitigate the need for tissue permeabilization optimization, e.g., by relying on the activity of RNase H enzyme to digest mRNA and release cDNA hybridized to it. Utilizing RNase H in the disclosed methods assay allows for no or mild permeabilization of the tissue sample (e.g., to allow the enzyme to enter the sample).

The present disclosure features methods, compositions, and kits for the spatial detection of target nucleic acids in a biological sample (e.g., a tissue section). Generally, spatial detection involves capture of a target nucleic acid, or proxy thereof, followed by one or more extension reactions. For example, other methods, including in situ reactions, can also be used for the spatial detection of analyte, or proxies thereof. Spatial detection of analytes includes a substrate including barcoded capture probes, where a capture probe on the substrate includes a spatial barcode (e.g., a nucleotide sequence unique to the location of the capture probe on the substrate) and a capture domain capable of hybridizing to a first adapter. The disclosed methods, compositions, and kits also provide the benefit of consistent permeabilization conditions and the capture of 5′ end proximal sequences of target nucleic acids.

Thus, provided herein are methods for determining a location of a target RNA in a biological sample, the method including: a) hybridizing a primer to the target RNA in the biological sample; b) extending (e.g., extending with a polymerase such as a reverse transcriptase) the primer using the target RNA as a template to provide (e.g., generate) a cDNA hybridized to the target RNA, thereby generating a cDNA:RNA duplex; c) incorporating (e.g., adding) at least three untemplated nucleotides (e.g., 5′-CCC-3′, 5′-CGC-3′) at a 3′ end of the cDNA of the cDNA:RNA duplex; d) hybridizing a first adapter to the at least three untemplated nucleotides and extending the cDNA of the cDNA:RNA duplex using the first adapter as a template, thereby generating an extended cDNA:RNA duplex; c) contacting a transposome complex with the biological sample to insert a second adapter into the extended cDNA:RNA duplex, thereby generating a 5′ fragmented cDNA:RNA duplex; f) releasing (e.g., digesting) the

RNA from the 5′ fragmented cDNA:RNA duplex, thereby generating a 5′ cDNA molecule including a complement of the first adapter and the second adapter; g) hybridizing the first adapter of the 5′ cDNA molecule to a capture domain of a capture probe on a substrate including a plurality of capture probes, where the capture probe includes: (i) a spatial barcode (as defined herein) and (ii) a capture domain (e.g., any of the capture domains described herein); and h) determining the sequence of (i) the spatial barcode, or a complement thereof, (ii) the 5′ cDNA molecule or a complement thereof, and using the determined sequences of (i) and (ii) to determine the location of the target RNA in the biological sample.

Also provided herein are methods for processing a target RNA in a biological sample, the method including: a) hybridizing a primer to the target RNA in the biological sample; b) extending (e.g., extending with a polymerase such as a reverse transcriptase) the primer using the target RNA as a template to provide a cDNA hybridized to the target RNA, thereby generating a cDNA:RNA duplex; c) incorporating (e.g., adding) at least three untemplated nucleotides at a 3′ end of the cDNA of the cDNA:RNA duplex; d) hybridizing a first adapter to the at least three untemplated nucleotides and extending the cDNA of the cDNA:RNA duplex using the first adapter as a template, thereby generating an extended cDNA:RNA duplex; e) contacting a transposome complex with the biological sample to insert a second adapter into the extended cDNA:RNA duplex, thereby generating a 5′ fragmented cDNA:RNA duplex; and f) releasing the RNA from the 5′ fragmented cDNA:RNA duplex, thereby generating a 5′ cDNA molecule including a complement of the first adapter and the second adapter.

Also disclosed herein are methods for determining a location of a target RNA in a biological sample, the method including: a) hybridizing a primer to the target RNA in the biological sample; b) extending (e.g., extending with a polymerase such as a reverse transcriptase) the primer using the target RNA as a template to provide (e.g., generate) a cDNA hybridized to the target RNA, thereby generating a cDNA:RNA duplex; c) incorporating (e.g., adding) at least three untemplated nucleotides (e.g., 5′-CCC-3′,5′-CGC-3′) at a 3′ end of the cDNA of the cDNA:RNA duplex; d) hybridizing a first adapter to the at least three untemplated nucleotides and extending the cDNA of the cDNA:RNA duplex using the first adapter as a template, thereby generating an extended cDNA:RNA duplex; e) contacting a transposome complex with the biological sample to insert a second adapter at one or more locations in the extended cDNA:RNA duplex. In some embodiments, the method includes f) releasing (e.g., digesting) the RNA from the extended cDNA:RNA duplex. In some embodiments, the method includes generating a 5′ cDNA molecule including a complement of the first adapter and the second adapter; a 3′ cDNA molecule, one or more middle cDNA molecules, or a combination thereof.

In some embodiments, the method includes hybridizing one or more of the generated fragments to a substrate including a plurality of capture probes, where a capture probe of the plurality includes: (i) a spatial barcode (as defined herein) and (ii) a capture domain (e.g., any of the capture domains described herein); and h) determining the sequence of (i) the spatial barcode, or a complement thereof, (ii) a sequence of the one or more generated fragments or a complement thereof, and using the determined sequences of (i) and (ii) to determine the location of the target RNA in the biological sample.

In some embodiments, the biological sample is disposed on the substrate including the capture probes. In some embodiments, the biological sample is disposed on a first substrate. In some embodiments, the method includes aligning the first substrate including the biological sample with a second substrate including the spatially-barcoded capture probes, such that at least a portion of the biological sample is aligned with at least a portion of the second substrate.

In some embodiments, the method includes migrating the 5′ cDNA molecule, the 3′ cDNA molecule, and/or the one or more middle cDNA molecules from the biological sample to the spatially-barcoded capture probes, and optionally, where the migrating includes electrophoresis.

In some embodiments, the primer hybridizes to a poly(A) sequence present in a target RNA (e.g., mRNA). In some embodiments, the primer hybridizes to target specific sequence in a target RNA.

In some embodiments, the capture probe can include one or more functional domains, and/or a cleavage domain. A functional domain typically includes a functional nucleotide sequence for a downstream analytical step in the overall analysis procedure. In some embodiments, the functional domain can include a sequencing specific site or a primer binding site. In some embodiments, the functional domain can include an amplification (e.g., PCR) sequence. In some embodiments, a capture probe includes a unique molecular identifier (UMI) as described herein. In some embodiments, the UMI is located 5′ to the capture domain in the capture probe.

In some embodiments, the extending the primer includes use of a reverse transcriptase (e.g., any of the reverse transcriptases described herein). In some embodiments, incorporating (e.g., adding) the at least three untemplated nucleotides includes use of a reverse transcriptase. In some embodiments, incorporating (e.g., adding) the at least three untemplated nucleotides includes use of a terminal transferase. In some embodiments, the terminal transferase is a terminal deoxynucleotidyl transferase (TdT). In some embodiments, the extending in step (d) includes use of a polymerase. In some embodiments, the polymerase is a DNA polymerase. In some embodiments, the polymerase is an RNA dependent DNA polymerase.

In some embodiments, the at least three untemplated nucleotides include a homopolynucleotide sequence (e.g., 5′-CCC-3′). In some embodiments, the at least three untemplated nucleotides include a heteropolynucleotide sequence (5′-CGC-3′).

In some embodiments, the first adapter is or includes RNA. In some embodiments, the first adapter is a template switching oligonucleotide. In some embodiments, the first adapter includes ribonucleotides such that the ribonucleotides can be digested after extension, thereby releasing the cDNA from any of the cDNA:RNA duplexes described herein from the target nucleic acid. In some embodiments, digesting the RNA from the cDNA:RNA duplexes includes use of an enzyme. In some embodiments, the enzyme is an endoribonuclease. In some embodiments, the endoribonuclease is one or more of RNase A, RNase C, RNase H, or RNase I. In some embodiments, the endoribonuclease includes RNase H.

RNase H is an endoribonuclease that specifically hydrolyzes the phosphodiester bonds of RNA, when hybridized to DNA. RNase H is part of a conserved family of ribonucleases which are present in many different organisms. There are two primary classes of RNase H: RNase H1 and RNase H2. Retroviral RNase H enzymes are similar to the prokaryotic RNase H1. All of these enzymes share the characteristic that they are able to cleave the RNA component of an RNA:DNA heteroduplex.

In some embodiments, the RNase H is RNase H1, RNase H2, or RNase H1, or RNase H2. In some embodiments, the RNase H includes but is not limited to RNase HII from Pyrococcus furiosus, RNase HII from Pyrococcus horikoshi, RNase HI from Thermococcus litoralis, RNase HI from Thermus thermophilus, RNase HI from E. coli, or RNase HII from E. coli.

In some embodiments, releasing or digesting the RNA includes denaturation. In some embodiments, denaturation includes use of heat or potassium hydroxide (KOH).

In some embodiments, a capture probe in the plurality of capture probes includes in a 5′ to a 3′ direction, a spatial barcode and a capture domain. In some embodiments, the capture probe includes one or more functional domains, a unique molecular identifier (UMI), a cleavage domain, or combinations thereof. In some embodiments, the capture domain is substantially complementary to the first adapter or a portion thereof. In some embodiments, the capture domain includes the sequence of the first adapter or a portion thereof. For example, the capture domain and the first adapter can be any sequence as long as the sequences are substantially complementary to one another to facilitate hybridization. In some embodiments, the substrate includes a plurality of capture probes collectively including a plurality of different capture domains.

In some embodiments, the second adapter sequences include a functional domain. In some embodiments, the functional domain includes a primer binding site. In some embodiments, the primer binding site, is a sequencing primer binding site.

In some embodiments, the substrate includes one or more features. In some embodiments, features are directly or indirectly attached or fixed to a substrate. In some embodiments, the features are not directly or indirectly attached or fixed to a substrate, but instead, for example, are disposed within an enclosed or partially enclosed three dimensional space (e.g., wells or divots). For example, the plurality of capture probes can be located on features on a substrate. In some embodiments, features include, but are not limited to, a spot, an inkjet spot, a masked spot, a pit, a post, a well, a ridge, a divot, a hydrogel pad, and a bead (e.g., a hydrogel bead). In some embodiments, a substrate includes a plurality of beads. For example, a substrate can include a monolayer of beads where each bead occupies a unique position on the substrate. In some instances, the beads can be immobilized on the substrate and can each contain a plurality of capture probes. In some instances, the capture probes on a particular bead have the same barcode, which is unique, and thus differs from the barcodes of capture probes on other beads. Thus, the barcode contained by the capture probes on each bead can serve as a spatial barcode that is associated with a distinct position on the substrate.

In some embodiments, the method includes permeabilizing the biological sample. Permeabilization of a biological sample can occur on a substrate where the substrate is aligned with the substrate such that at least a portion of the biological sample is aligned with at least a portion of the substrate. In some embodiments, permeabilization of the biological sample occurs directly on a substrate including a plurality of capture probes. In some embodiments, permeabilization of the biological sample occurs in a sandwich configuration as described herein (e.g., a first substrate including the biological sample brought into proximity of or contact with a second substrate including a substrate of capture probes). In some embodiments, the permeabilizing includes use of a protease. In some embodiments, the protease includes one or more of pepsin, proteinase K, and collagenase. In some embodiments, the protease includes Proteinase K.

In some embodiments, the biological sample is fixed. In some embodiments, the biological sample is methanol-fixed, acetone-fixed, paraformaldehyde-fixed, or is formalin-fixed paraffin-embedded (FFPE).

In some embodiments, the FFPE sample or section is deparaffinized, permeabilized, equilibrated, and blocked before the in situ reverse transcription reaction occurs. In some embodiments, deparaffinization includes using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water.

In some embodiments, the method includes staining the biological sample. In some embodiments, the biological sample is stained after fixation. In some embodiments, the biological sample is stained before fixation. In some embodiments, the staining includes optical labels as described herein, including, but not limited to, fluorescent (e.g., fluorophore), radioactive (e.g., radioisotope), chemiluminescent (e.g., a chemiluminescent compound), a bioluminescent compound, calorimetric, or colorimetric detectable labels. In some embodiments, the staining includes a fluorescent antibody directed to a target analyte (e.g., cell surface or intracellular proteins) in the biological sample. In some embodiments, the staining includes an immunohistochemistry stain directed to a target analyte (e.g., cell surface or intracellular proteins) in the biological sample. In some embodiments, the staining includes a chemical stain, such as hematoxylin and cosin (H&E) or periodic acid-Schiff (PAS). In some embodiments, staining the biological sample includes the use of a biological stain including, but not limited to, acridine orange, Bismarck brown, carmine, coomassie blue, cresyl violet, DAPI, cosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In some embodiments, significant time (e.g., days, months, or years) can clapse between staining and/or imaging the biological sample.

In some embodiments, the method includes imaging the biological sample. In some embodiments, the biological sample (e.g., a tissue section) is imaged after fixation. In some embodiments, the biological sample is imaged before fixation. In some embodiments, imaging includes one or more of expansion microscopy, bright field microscopy, dark field microscopy, phase contrast microscopy, electron microscopy, fluorescence microscopy, reflection microscopy, interference microscopy and confocal microscopy.

The methods disclosed herein can be performed on any type of biological sample. In some embodiments, the biological sample is a tissue sample. In some embodiments, the tissue sample is a fixed tissue sample. In some embodiments, the fixed tissue sample is a methanol-fixed tissue sample, an acetone-fixed tissue sample, a paraformaldehyde tissue sample, or a formalin-fixed paraffin-embedded tissue sample. In some embodiments, the tissue sample is a fresh-frozen tissue sample. In some embodiments, the biological sample is a tissue section. In some embodiments, the tissue section is a fixed tissue section. In some embodiments, the fixed tissue section is a methanol-fixed tissue section, an acetone-fixed tissue section, a paraformaldehyde tissue section, or a formalin-fixed paraffin-embedded tissue section. In some embodiments, the tissue section is a fresh-frozen tissue section.

In some embodiments, the methods are used to analyze RNA. Non-limiting examples of RNA include various types of coding and non-coding RNA. Examples of the different types of RNA analytes include messenger RNA (mRNA), ribosomal RNA (rRNA), transfer RNA (RNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA). The RNA can be from an RNA virus, for example RNA viruses from Group III, IV or V of the Baltimore classification system. The RNA can be from a retrovirus, such as a virus from Group VI of the Baltimore classification system.

In some embodiments, the determining step includes sequencing. In some embodiments, the sequencing includes high-throughput sequencing.

### i. Tagmentation

Transposase enzymes and transposons can be utilized in methods of spatial analysis. Generally, transposition is the process by which a specific genetic sequence (e.g., a transposon sequence) is relocated from one place in a genome to another. Many transposition methods and transposable elements are known in the art (e.g., DNA transposons, retrotransposons, autonomous transposons, non-autonomous transposons). One non-limiting example of a transposition event is conservative transposition. Conservative transposition is a non-replicative mode of transposition in which the transposon is completely removed from the genome and reintegrated into a new locus, such that the transposon sequence is conserved, (e.g., a conservative transposition event can be thought of as a “cut and paste” event) (See, e.g., Griffiths A. J., et. al., Mechanism of transposition in prokaryotes. An Introduction to Genetic Analysis (7th Ed.). New York: W. H. Freeman (2000)).

In one example, cut and paste transposition can occur when a transposase enzyme binds a sequence flanking the ends of the transposon (e.g., a recognition sequence, e.g., a mosaic end sequence, a transposon sequence). A transposome (e.g., a transposition complex) forms and the endogenous DNA can be manipulated into a pre-excision complex such that two transposase enzymes can interact. In some embodiments, when the transposases interact double stranded breaks are introduced into the DNA or DNA:RNA duplexes (e.g., cDNA:RNA duplexes; See Di, L., et al, RNA sequencing by direct tagmentation of RNA:DNA hybrids, Applied Biological Sciences, 117 (6) 2886-2893 (2020)). The transposase enzymes can locate and bind a target site in the DNA, create a double stranded break, and insert the transposon end sequence (See, e.g., Skipper, K. A., et. al., DNA transposon-based gene vehicles-scenes from an evolutionary drive, J Biomed Sci., 20:92 (2013)).

Transposome-mediated fragmentation and tagging (“tagmentation”) is a process of transposase-mediated fragmentation and tagging of DNA or RNA:DNA duplexes (e.g., hybrids). A transposome is a complex of a transposase enzyme and DNA which comprises a transposon end sequence (also known as “transposase recognition sequence” or “mosaic end” (MEs)). In some methods of spatial analysis, RNA:DNA duplexes are fragmented in such a manner that a second adapter is inserted into the fragmented RNA:DNA (e.g., the fragmented RNA:DNA is “tagged”). In some embodiments, the second adapter includes a functional sequence. The functional sequence can include a sequencing specific site or a primer binding site.

A transposase dimer (e.g., in the case of Tn5 transposase system) in conjunction with a transposon sequence (e.g., forming a complex called a transposome) is able to simultaneously fragment RNA:DNA based on its transposon recognition sequences and ligate DNA from the transposome (e.g., transposon sequence) to the fragmented RNA:DNA (e.g., tagmented RNA:DNA). This system has been adapted using hyperactive transposase enzymes and modified DNA molecules (adaptors) comprising MEs to fragment RNA:DNA and tag both strands of RNA:DNA (e.g., RNA:cDNA) duplex fragments with functional DNA molecules (e.g., primer binding sites). In particular embodiments, the Tn5 transposase may be produced as purified protein monomers. Tn5 transposase is also commercially available (e.g., manufacturer Illumina, Illumina.com, Catalog No. 15027865, TD Tagment DNA Buffer Catalog No. 15027866). These can be subsequently loaded with the oligonucleotides of interest, e.g., ssDNA oligonucleotides containing MEs (e.g., transposon sequences) for Tn5 recognition and additional functional sequences (e.g., primer binding sites) are annealed to form a dsDNA mosaic end oligonucleotide (MEDS) that is recognized by Tn5 during dimer assembly (e.g., transposome dimerization). In some embodiments, a hyperactive Tn5 transposase can be loaded with adapters which can simultaneously fragment and tag RNA:DNA duplexes.

In some embodiments, the step of fragmenting the RNA:DNA duplexes in the biological sample comprises contacting the biological sample containing the RNA:DNA duplexes with the transposase enzyme (e.g., a transposome, e.g., a reaction mixture (e.g., solution)) including a transposase), under any suitable condition. In some embodiments, such suitable conditions result in the tagmentation of the RNA:DNA (e.g., RNA:cDNA) duplexes generated by the in situ reverse transcription reaction described herein. Suitable conditions can be conditions (e.g., buffer, salt, concentration, pH, temperature, time conditions) under which the transposase enzyme is functional, e.g., in which the transposase enzyme displays transposase activity, particularly tagmentation activity, in the biological sample.

The term “functional”, as used herein in reference to transposase enzymes, is meant to include embodiments in which the transposase enzyme can show some reduced activity relative to the activity of the transposase enzyme in conditions that are optimum for the enzyme, e.g., in the buffer, salt and temperature conditions recommended by the manufacturer. Thus, the transposase can be considered to be “functional” if it has at least about 50%, e.g., at least about 60%, about 70%, about 80%, about 85%, about 90%, about 95%, about 96%, about 97%, about 98%, about 99%, or about 100%, activity relative to the activity of the transposase in conditions that are optimum for the transposase enzyme.

In some embodiments, the transposase enzyme of the transposome complex is a Tn5 transposase, or a functional derivate or variant thereof. (See, e.g., Reznikoff et al, WO 2001/009363, U.S. Pat. Nos. 5,925,545, 5,965,443, 7,083,980, and 7,608,434, and Goryshin and Reznikoff, J. Biol. Chem. 273:7367, (1998), which are herein incorporated by reference). In some embodiments, the Tn5 transposase is a hyper Tn5 transposase, or a functional derivate or variate thereof (U.S. Pat. No. 9,790,476, incorporated herein by reference). For example, the Tn5 transposase can be a fusion protein (e.g., a Tn5 fusion protein). Tn5 is a member of the RNase superfamily of proteins. The Tn5 transposon is a composite transposon in which two near-identical insertion sequences (IS50L and IS50R) flank three antibiotic resistance genes. Each IS50 contains two inverted 19-bp end sequences (ESs), an outside end (OE) and an inside end (IE). Wild-type Tn5 transposase enzyme is generally inactive (e.g., low transposition event activity). However, amino acid substitutions can result in hyperactive variants or derivatives. In one non-limiting example, amino acid substitution, L372P, substitutes a leucine amino acid for a proline amino acid which results in an alpha helix break, thus inducing a conformational change to the C-terminal domain. The alpha helix break separates the C-terminal domain and N-terminal domain sufficiently to promote higher transposition event activity (See, Reznikoff, W.S., Tn5 as a model for understanding DNA transposition, Mol Microbiol, 47(5): 1199-1206 (2003)). Other amino acid substitutions resulting in hyperactive Tn5 are known in the art. For example, the improved avidity of the modified transposase enzyme (e.g., modified Tn5 transposase enzyme) for the repeat sequences for OE termini (class (1) mutation) can be achieved by providing a lysine residue at amino acid 54, which is glutamic acid in wild-type Tn5 transposase enzyme (See U.S. Pat. No. 5,925,545). The mutation strongly alters the preference of the modified transposase enzyme (e.g., modified Tn5 transposase enzyme) for OE termini, as opposed to IE termini. The higher binding of this mutation, known as EK54, to OE termini results in a transposition rate that is about 10-fold higher than is seen with wild-type transposase enzyme (e.g., wild type Tn5 transposase enzyme). A similar change at position 54 to valine (e.g., EV54) also results in somewhat increased binding/transposition for OE termini, as does a threonine to proline change at position 47 (e.g., TP47; about 10-fold higher) (See U.S. Pat. No. 5,925,545). Any transposase enzyme with tagmentation activity, e.g., any transposase enzyme capable of fragmenting RNA:DNA duplexes and inserting second adapters to the ends of the fragmented (e.g., tagmented) cDNA, can be used. In some embodiments, the transposase is any transposase capable of conservative transposition. In some embodiments, the transposase is a cut and paste transposase. Other kinds of transposases are known in the art and are within the scope of this disclosure. For example, suitable transposase enzymes include, without limitation, Mos-1, HyperMu™, Ts-Tn5, Ts-Tn5059, Hermes, Tn7, a Vibrio species transposase (See e.g., U.S. patent application Ser. No. 20/120,301925A1 and WO 2015/069374, the contents of which are herein incorporated by reference in their entireties), or any functional variant or derivative of the previously listed transposase enzymes.

After in situ reverse transcription and addition of the first adapter to the 3′ end of the cDNA of the cDNA/RNA duplex, the biological sample can be contacted with a plurality of transposome complexes. The transposome complex inserts second adapters as described herein via ligation and thereby generates different pluralities of fragmented cDNA:RNA duplexes. For example, the fragments include a plurality of 5′ fragmented cDNA:RNA duplexes.

The following describes capture of the cDNA molecule of the 5′ fragmented cDNA:RNA duplexes (e.g., 5′ cDNA molecule capture). The cDNA of the 5′ fragmented cDNA:RNA duplex includes in a 5′ to 3′ direction the second adapter sequence (e.g., a functional sequence), the transposon end sequence (e.g., mosaic end), a complement of the target nucleic acid, the incorporated untemplated nucleotides, and the first adapter sequence. In some embodiments, the RNA is released (e.g., digested, denatured) from the cDNA of the fragmented cDNA:RNA duplexes, thereby generating a 5′ cDNA molecule. The 5′ cDNA molecule is a partially double-stranded nucleic acid product that includes a DNA sequence complementary to the transposon end sequence. In some embodiments, the first adapter is a sequence complementary to, or having the same sequence as, the capture domain of a capture probe on the substrate. In some embodiments, the first adapter is a template switching oligonucleotide. In some embodiments, the template switching oligonucleotide is an RNA oligonucleotide.

The transposome complexes also generate additional pluralities of fragmented RNA:DNA duplexes including 3′ fragmented cDNA:RNA duplexes and one or more middle fragmented cDNA:RNA duplexes after in situ reverse transcription. Capture of not only the 5′ cDNA molecules, but also the 3′ cDNA molecules, and the one or more middle cDNA molecules is useful for determining gene expression in a spatial context.

The cDNA of the 3′ fragmented cDNA:RNA duplex includes in a 5′ to 3′ direction a poly(T) sequence and a transposon end sequence (e.g., a mosaic end sequence). In some embodiments, there is a gap between the poly(T) sequence and the mosaic end sequence that can be introduced during transposition depending on the transposase enzyme used. Next, the RNA can be released (e.g., digested, denatured) from the 3′ fragmented cDNA:RNA duplex resulting in two fragments. The first fragment is a poly(T) sequence generated by the reverse transcription reaction (which may not be captured) and the second fragment is a partially double-stranded product where the cDNA of the cDNA:RNA duplex includes the transposon end sequence hybridized to a complementary sequence. The complementary sequence e.g., the “RNA” strand includes single-stranded portions of RNA (e.g., the target nucleic acid) and the second adapter. The single-stranded RNA remains intact after digestion since the RNase only degrades RNA hybridized to DNA. Next, a second round of in situ reverse transcription and/or gap-filling or extension reactions can be performed that reverse transcribes the target nucleic acid sequence between the poly(T) sequence and the transposon end sequence and also incorporates a complement of the second adapter. The resulting cDNA product includes in a 5′ to 3′ direction a sequence complementary to the target nucleic acid, a transposon end sequence, and a second adapter sequence. A second population of capture probes on the substrate can include a capture domain that is complementary to all or a portion of the sequence of the transposon end sequence and/or the second adapter.

In some embodiments, the second round of reverse transcription occurs before the RNA is released from the 3′ fragmented cDNA:RNA duplex. In such embodiments, the 3′ cDNA molecule in a 5′ to 3′ direction a poly(T) sequence, a sequence complementary to the target nucleic acid, a transposon end sequence, and a second adapter sequence. A second population of capture probes on the substrate can include a capture domain that is complementary to all or a portion of the sequence of the transposon end sequence and/or the second adapter.

Further, a plurality of middle fragmented cDNA:RNA duplexes can be generated after in situ reverse transcription and fragmentation with the transposome complexes. For example, the cDNA of a middle fragmented cDNA:RNA duplex includes in a 5′ to 3′ direction a second adapter, a transposon end sequence (e.g., a mosaic end sequence), a sequence complementary to the target nucleic acid, and a second transposon end sequence (e.g., a mosaic end sequence). In some embodiments, there is a gap between the sequence complementary to the target nucleic acid and the second mosaic end sequence that can be introduced during transposition depending on the transposase enzyme used. Next, the RNA can be released (e.g., digested, denatured) from the 3′ fragmented cDNA:RNA duplex resulting in two fragments. The first fragment includes a partially double-stranded product where the one or more middle cDNA molecules includes the transposon end sequence hybridized to a complementary sequence. The complementary sequence e.g., the “RNA” strand includes single-stranded portions of RNA (e.g., the target nucleic acid) and the second adapter. The single-stranded RNA remains intact after digestion since the RNase only degrades RNA hybridized to DNA. The second fragment includes a partially double-stranded product where the cDNA of the second fragment includes in a 5′ to 3′ direction a second adapter sequence, a transposon end sequence (e.g., a mosaic end sequence), and a sequence complementary to the target nucleic acid. Next, a second round of in situ reverse transcription and/or gap-filling or extension reactions can be performed that reverse transcribe the target nucleic acid of the first fragment and an extension reaction incorporating a sequence complementary to the second adapter. The second fragment can be captured by a second population of capture probes on the substrate can include a capture domain that is complementary to all or a portion of the sequence of the transposon end sequence and/or the second adapter without the need for a second round of in situ reverse transcription.

In some embodiments, the second round of reverse transcription occurs before the RNA is released from the one or more middle fragmented cDNA:RNA duplexes. In such embodiments, the one or more middle cDNA molecules includes in a 5′ to 3′ direction a second adapter sequence, a transposon end sequence (e.g., a mosaic end), a sequence complementary to the target nucleic acid sequence, another transposon end sequence, and another second adapter sequence. A second population of capture probes on the substrate can include a capture domain that is complementary to all or a portion of the sequence of the transposon end sequence and/or the second adapter sequence.

In some embodiments, the transposome complex includes a transposase enzyme, a transposon sequence, and the second adapter; and optionally where the transposase enzyme is a Tn5 transposase enzyme, a Mu transposase enzyme, a Tn7 transposase enzyme, a Vibrio species transposase, or functional derivatives thereof.

In some embodiments, the Tn5 transposase enzyme, or functional variant or derivative thereof, comprises an amino acid sequence having at least 80% sequence identity to SEQ ID NO: 1. In some embodiments, the Tn5 transposase enzyme, or functional variant or derivative thereof, comprises an amino acid sequence having a sequence identity of at least about 90%, 91%, 92%, 93%, 94%, 95%, 96%, 97%, 98%, 99%, or 100% amino acid sequence identity to SEQ ID NO: 1. In some embodiments, the transposase enzyme is complexed with an adapter including a transposon end sequence. In some embodiments, the Tn5 transposon end sequence comprises a sequence having at least 80% sequence identity to SEQ ID NO: 2. In some embodiments, the Tn5 transposon end sequence comprises an amino acid sequence having a sequence identity of at least about 90%, 91%, 92%, 93%, 94%, 95%, 96%, 97%, 98%, 99%, or 100% amino acid sequence identity to SEQ ID NO. 2.

In some embodiments, the transposase enzyme is a Mu transposase enzyme, or a functional variant or derivative thereof. In some embodiments, the Mu transposase enzyme, or functional variant or derivative thereof, comprises an amino acid sequence having at least 80% identity to SEQ ID NO: 3. In some embodiments, the Mu transposase enzyme, or functional variant or derivative thereof, comprises an amino acid sequence having a sequence identity of at least about 90%, 91%, 92%, 93%, 94%, 95%, 96%, 97%, 98%, 99%, or 100% amino acid sequence identity to SEQ ID NO: 3. In some embodiments, the Mu transposon end sequence (e.g., a transposase recognition sequence) comprises a sequence having at least 80% sequence identity to any one of SEQ ID NOs: 4-9. In some embodiments, the Mu transposon end sequence (e.g., a Mu transposase recognition sequence) comprises a sequence having at least about 90%, 91%, 92%, 93%, 94%, 95%, 96%, 97%, 98%, 99%, or 100% amino acid sequence identity to any one of SEQ ID NOs: 4-9.

In some embodiments, the transposase enzyme is an ISR family transposase, or a functional variant or derivative thereof. For example, the ISR family transposase can be an ISR family transposase described in NCBI Reference Sequence: WP_012128611.1 and/or U.S. Pat. No. 9,005,935, which is incorporated herein by reference in its entirety. In some embodiments, the ISR family transposase, or functional variant or derivative thereof, comprises an amino acid sequence having at least 80% identity to SEQ ID NO: 10. In some embodiments, the ISR family transposase, or functional variant or derivative thereof, comprises an amino acid sequence having a sequence identity of at least about 90%, 91%, 92%, 93%, 94%, 95%, 96%, 97%, 98%, 99%, or 100% amino acid sequence identity to SEQ ID NO: 10. In some embodiments, the ISR family transposase transposon end sequence (e.g., transposase recognition sequence) comprises a sequence having at least 80% identity to any one of SEQ ID NOs: 11-13. In some embodiments, the ISR family transposase transposon end sequence (e.g., transposase recognition sequence) comprises a sequence having at least about 90%, 91%, 92%, 93%, 94%, 95%, 96%, 97%, 98%, 99%, or 100% amino acid sequence identity to SEQ ID NOs: 11-13.

In some embodiments, the method includes hybridizing the second adapter of: (i) the 3′ cDNA molecule to the second capture domain, and (ii) the one or more middle fragmented cDNA molecule(s) to another capture domain, e.g., different second capture domain(s) or a third capture domain.

In some embodiments, the method includes extending the capture probe using the 5′ cDNA molecule as a template (e.g., thereby generating an extended capture probe). In some embodiments, the method includes extending the 5′ cDNA molecule using the capture probe as a template (e.g., thereby generating an extended 5′ cDNA).

In some embodiments, the method includes extending the second capture probe using the 3′ cDNA molecule and/or one of the one or more middle cDNA molecule(s) as a template (e.g., thereby generating an extended second capture probe).

In some embodiments, the method includes extending the 3′ cDNA molecule and/or one of the one or more middle cDNA molecule(s) using the second capture probe as a template (e.g., thereby generating an extended 3′ CDNA molecule and/or one or more extended middle cDNA molecule(s)).

In some embodiments, the methods involve amplifying (e.g., via polymerase chain reaction) an extended capture probe or a complement thereof, an extended second capture probe or a complement thereof, an extended 5′ cDNA or a complement thereof, an extended 3′ cDNA or a complement thereof, an extended middle cDNA molecule or a complement thereof, or a combination thereof.

In some embodiments, the method includes determining the sequence of: (i) the second spatial barcode, or a complement thereof, and (ii) the 3′ cDNA molecule, or a complement thereof, and using the determined sequences of (i) and (ii) to determine the location of the target RNA in the biological sample.

In some embodiments, the method includes determining the sequence of: (i) the second spatial barcode, or a complement thereof, and (ii) the one or more middle cDNA molecule(s), or complement(s) thereof, and using the determined sequences of (i) and (ii) to determine the location of the target RNA in the biological sample. In any of the foregoing determining steps, the determining can be performed by sequencing an extended capture probe or a complement thereof, an extended second capture probe or a complement thereof, an extended 5′ cDNA or a complement thereof, an extended 3′ cDNA or a complement thereof, an extended middle cDNA molecule or a complement thereof, or a combination thereof.

### ii. Kits

The present disclosure also features kits for spatial detection of target nucleic acids. For example, also provided herein are kits including: (a) a substrate including a plurality of capture probes, where a capture probe of the plurality of capture probes includes: (i) a spatial barcode and (ii) a capture domain; (b) a plurality of transposome complexes; (c) a plurality of primers complementary to a plurality of target RNAs; (d) a plurality of first adapters; and (e) instructions for performing any of the methods described herein.

In some embodiments, the kit includes one or more permeabilization reagents. In some embodiments, the one or more permeabilization reagents includes one or more proteases, a DNase, an RNase, a lipase, a detergent, or combinations thereof. In some embodiments, the one or more proteases include pepsin, proteinase K, or collagenase.

In some embodiments, the capture probe includes one or more functional domains, a cleavage domain, a unique molecular identifier (UMI), or combinations thereof. In some embodiments, the one or more functional domains includes a primer binding site or a sequencing specific site.

In some embodiments, the kit includes a polymerase. In some embodiments, the polymerase includes a reverse transcriptase and/or a DNA polymerase. In some embodiments, the kit includes a plurality of dNTPs (e.g., dATPs, dTTPs, dCTPs, dGTPs).

In some embodiments, a transposome complex of the plurality of transposome complexes includes a transposase enzyme, a transposon sequence, and a second adapter. In some embodiments, the transposase enzyme is a Tn5 transposase enzyme, a Mu transposase enzyme, a Tn7 transposase enzyme, a Vibrio species transposase, or functional derivatives thereof.

In some embodiments, a first adapter of the plurality of first adapters is a template switch oligonucleotide.

### iii. Compositions

In addition to the methods and kits described herein, the present disclosure also provides for compositions related to any of the target nucleic acid detection methods described herein.

Thus, provided herein are compositions including: (a) a substrate including a plurality of capture probes (e.g., any of the capture probes described herein), where a capture probe of the plurality of capture probes includes: (i) a spatial barcode and (ii) a capture domain (e.g., any of the capture domains described herein); (b) a plurality of transposome complexes; (c) a plurality of primers complementary to a plurality of target RNAs; and (d) a plurality of first adapters.

Also provided herein are composition including: (a) a substrate including a plurality of capture probes, where a capture probe of the plurality of capture probes includes: (i) a spatial barcode and (ii) a capture domain; (b) a plurality of extended primers (e.g., primers hybridized to target nucleic acids and that have been extended e.g., extended with a reverse transcriptase or a polymerase); and (c) a plurality of cDNA:RNA duplexes.

Also provided herein are compositions including: (a) a substrate including a plurality of capture probes, where a capture probe of the plurality of capture probes includes: (i) a spatial barcode and (ii) a capture domain; (b) a plurality of transposome complexes; and (c) a plurality of 5′ end fragmented cDNA:RNA duplexes, a plurality of 3′ end fragmented cDNA:RNA duplexes, and/or one or more middle fragmented cDNA:RNA duplexes. Also provided herein are compositions including: (a) a substrate including a plurality of capture probes, where a capture probe of the plurality of capture probes includes: (i) a spatial barcode and (ii) a capture domain; and (b) a plurality of 5′ end fragmented cDNA:RNA duplexes, where the cDNA of the 5′ end fragmented cDNA:RNA duplex includes a first adapter at the 3′ end. For example, the plurality of 5′ end fragmented cDNA:RNA molecules result from the extension of primers hybridized to the RNA (e.g., mRNA) in situ. Additional untemplated nucleotides (e.g., a heteropolynucleotide sequence, a homopolynucleotide sequence) can be incorporated (e.g., added) to the cDNA of the cDNA:RNA duplex. The first adapter can hybridize to the untemplated nucleotides added to the 3′ of the cDNA molecule.

Also provided herein are compositions including: (a) a substrate including a plurality of capture probes, where a capture probe of the plurality of capture probes includes: (i) a spatial barcode and (ii) a capture domain; and (b) a plurality of gap-filled 3′ end fragmented cDNA:RNA duplexes and one or more gap-filled middle fragmented cDNA:RNA duplexes. For example, after cDNA:RNA duplexes are generated in situ, they can be tagmented with a transposome complex. The transposome complex fragments (e.g., tagments) the cDNA:RNA duplexes into a plurality of 5′ end fragmented cDNA:RNA duplexes, a plurality of 3′ end fragmented cDNA:RNA duplexes, and one or more middle fragmented cDNA:RNA duplexes. Fragmenting via a transposome complex generates gaps in the fragments, therefore, in some embodiments, the gaps can be gap-filled via a polymerase reaction.

Also provided herein are composition including: (a) a substrate including a plurality of capture probes, where a capture probe of the plurality of capture probes includes: (i) a spatial barcode and (ii) a capture domain; (b) a plurality of extended 5′ end fragmented cDNA:RNA duplexes, a plurality of gap-filled 3′ end fragmented cDNA:RNA duplexes, and one or more gap-filled middle fragmented cDNA:RNA duplexes; and (c) an RNase. In some embodiments, the RNase releases (e.g., digests) the RNA from the any of the fragmented cDNA:RNA duplexes described herein.

Also provided herein are compositions including: (a) a substrate including a plurality of capture probes, where a capture probe of the plurality of capture probes includes: (i) a spatial barcode and (ii) a capture domain; (b) a plurality of 5′ cDNA molecules including a first adapter sequence; (c) a plurality of 3′ cDNA molecules including a second adapter sequence; and (d) one or more gap-filled middle fragmented cDNA molecules including a second adapter sequence.

Also provided herein are composition including: (a) a substrate including: (i) a plurality of capture probes, where a capture probe of the plurality of capture probes includes: (i) a spatial barcode and (ii) a capture domain; and (ii) a second plurality of capture probes, where a second capture probe of the second plurality of capture probes includes: (i) a second spatial barcode and (ii) a second capture domain; and (b) one or more of: (i) a plurality of 5′ cDNA molecules including a first adapter sequence hybridized to the capture domain of the capture probe; (ii) a plurality of 3′ cDNA molecules including a second adapter sequence hybridized to a second capture domain of a second capture probe; and (iii) one or more gap-filled middle fragmented cDNA molecules including a second adapter sequence hybridized to a different second capture domain of a second capture probe.

In some embodiments, the composition includes one or more permeabilization reagents. In some embodiments, the one or more permeabilization reagents includes one or more proteases, a DNase, an RNase, a lipase, a detergent, or combinations thereof. In some embodiments, the one or more proteases include pepsin, proteinase K, and collagenase.

In some embodiments, the capture probe and/or the second capture probe includes one or more functional domains, a cleavage domain, a unique molecular identifier (UMI) as described herein, or combinations thereof. In some embodiments, the one or more functional domains includes a primer binding site or a sequencing specific site.

In some embodiments, the composition includes a polymerase (e.g., any of the polymerases described herein). In some embodiments, the polymerase includes a reverse transcriptase and/or a DNA polymerase. In some embodiments, the composition includes a plurality of dNTPs (e.g., dATPs, dTTPs, dCTPs, dGTPs).

In some embodiments, a transposome complex of the plurality of transposome complexes includes a transposase enzyme, a transposon sequence, and an adapter. In some embodiments, the transposase enzyme is a Tn5 transposase enzyme, a Mu transposase enzyme, a Tn7 transposase enzyme, a Vibrio species transposase, or functional derivatives thereof.

In some embodiments, a first adapter of the plurality of first adapters is a template switch oligonucleotide.

### iv. Library Preparation

In some embodiments, the fragmented cDNA products captured on the substrate (e.g., the 5′ fragmented cDNA molecules, or a complement thereof, the 3′ fragmented cDNA molecules, or a complement thereof, the one or more middle fragmented cDNA molecules, or a complement thereof), and/or amplicons of such products, can be prepared for downstream applications, such as generation of a sequencing library and next-generation sequencing. Generating sequencing libraries are known in the art. For example, the fragmented cDNA products, or complements thereof, can be purified and collected for downstream amplification steps. The amplification products can be amplified using PCR, where primer binding sites flank the spatial barcode and products described above, or a complement thereof, generating a library associated with a particular spatial barcode. In some embodiments, the library preparation can be quantitated and/or quality controlled to verify the success of the library preparation steps. The library amplicons are sequenced and analyzed to decode spatial information of the crosslinked digested DNA.

Alternatively, or additionally, the amplicons can then be enzymatically fragmented and/or size-selected in order to provide for desired amplicon size. In some embodiments, when utilizing an Illumina® library preparation methodology, for example, P5 and P7, sequences can be added to the amplicons thereby allowing for capture of the library preparation on a sequencing flow cell (e.g., on Illumina sequencing instruments). Additionally, i7 and i5 can index sequences be added as sample indexes if multiple libraries are to be pooled and sequenced together. Further, Read 1 and Read 2 sequences can be added to the library for sequencing purposes, if not already present on the capture probe on the substrate and incorporated into the captured crosslinked and digested DNA. The aforementioned sequences can be added to a library preparation sample, for example, via End Repair, A-tailing, Adaptor Ligation, and/or PCR. The cDNA fragments can then be sequenced using, for example, paired-end sequencing using TruSeq Read 1 and TruSeq Read 2 as sequencing primer sites, although other methods are known in the art.

### EXAMPLES

### Example 1. 5′ Gene Expression via Transposome Mediated Spatial Capture

FIG. 12 (top) shows an example of an in situ reverse transcribed cDNA product hybridized to a target RNA. Briefly, the biological sample is contacted with a plurality of primers (e.g., poly(T) primers, target specific primers, etc.) that hybridize to the target nucleic acid analytes (e.g., target RNA) and are extended thereby generating cDNA hybridized to the target nucleic acid (e.g., a cDNA:RNA duplex). A template switching reaction occurs. For example, untemplated nucleotides (e.g., 5′-CCC-3′; 5′-CGC-3′) are incorporated (e.g., added) at the 3′ end of the cDNA (shown in FIG. 12 as 5′-CCC-3′). A first adapter (e.g., a template switching oligonucleotide) hybridizes to the untemplated nucleotides and the cDNA is further extended using the first adapter as a template in the 5′ fragment, thereby generating an extended cDNA:RNA duplex (having a complement of the first adapter).

The resulting cDNA:RNA duplex is contacted with a plurality of transposome complexes that can generate three different cDNA:RNA fragments also shown in FIG. 12 (e.g., a 5′ cDNA:RNA fragment, a 3′ cDNA:RNA fragment, and one or more middle cDNA:RNA fragments). The 5′ cDNA:RNA fragment, a 3′ cDNA:RNA fragment, and one or more middle cDNA:RNA fragments each also have one or more second adapter sequences. The transposome complex inserts second adapters as described herein via ligation and thereby generates different pluralities of fragmented cDNA:RNA duplexes. For example, the fragments include a plurality of 5′ fragmented cDNA:RNA duplexes. In some embodiments, the cDNA of the 5′ fragmented cDNA:RNA duplex includes in a 5′ to 3′ direction the second adapter sequence (e.g., a functional sequence (e.g., a Read2 sequence, a partial Read2 sequence), the transposon end sequence (e.g., mosaic end), a complement of all or a portion of the target nucleic acid, the incorporated untemplated nucleotides, and the first adapter sequence.

The RNA is released (e.g., digested, denatured, etc.) from the cDNA of the fragmented cDNA:RNA duplexes. FIG. 13 shows the resulting products after the RNA is released from each of the fragmented cDNA:RNA duplexes. For example, post-RNA release, the 5′ cDNA molecule is a partially double-stranded nucleic acid product that includes a DNA sequence complementary to the transposon end sequence as shown in FIG. 13 (top). The other post-RNA release products result in multiple fragments (e.g., 3′ fragments, middle fragments) as described herein are also shown in FIG. 13. FIG. 13 (bottom) shows capture of the 5′ cDNA molecule by a capture probe on a substrate that includes a capture domain complementary to the first adapter sequence (e.g., template switch oligonucleotide) of the 5′ cDNA molecule and barcode (e.g., a spatial barcode).

The capture probe and/or the captured 5′ cDNA molecule can be extended (e.g., extended by their 3′ ends, respectively) to generate an extended capture probe and/or an extended 5′ cDNA molecule. Either product can be released from the array, optionally amplified, and processed for library preparation as described herein to determine the location of the target nucleic acid (e.g., RNA) in the biological sample.

### Example 2. Full-Length Gene Expression via Transposome Mediated Spatial Capture

In some embodiments, full-length gene expression via transposome mediated spatial capture begins with in situ reverse transcription of target nucleic acids, followed by fragmentation via a plurality of transposome complexes as described in Example 1 above.

FIG. 14 is a schematic diagram showing the three different cDNA:RNA fragments of FIG. 12 (e.g., a 5′ fragmented cDNA:RNA duplex, a 3′ fragmented RNA:DNA duplex, and a middle fragmented RNA:DNA duplex) where reverse transcription and/or extension reactions have filled in gaps or extended missing sequences introduced during transposition (e.g., tagmentation). Thus, FIG. 14 shows double-stranded RNA:cDNA duplexes where the RNA and/or RNA:DNA hybridized to the cDNA molecules (e.g., 5′ cDNA molecules, 3′ cDNA molecules, and/or middle 3′ cDNA molecules) is released (not shown) (e.g., released via denaturation (e.g., via potassium hydroxide or heat), or digested) and hybridize to capture domains of capture probes on a substrate. In this Example, the substrate includes two populations of capture probes to facilitate capture of the three different cDNA molecules. For example, capture of the 5′ cDNA molecule includes use of a capture probe with a capture domain complementary to the first adapter added during reverse transcription. Capture of the 3′ cDNA molecules and the one or more middle cDNA molecules includes a capture probe with a capture domain complementary to all or a portion of the second adapter (e.g., added during tagmentation) and/or the transposon end sequence (e.g., a mosaic end sequence).

As described above the captured cDNA molecules (e.g., the 5′ cDNA molecule, the 3′ cDNA molecule, and/or the one or more middle cDNA molecules) can be extended using the capture probe as a template, thereby generating extended cDNA molecules. Conversely, the capture probe (e.g., the capture probe including a capture domain that hybridizes to the 5′ cDNA molecule and/or the capture second probe including a capture domain that hybridizes to all or a portion of the second adapter and/or the transposon end sequence) is extended, thereby generating extended capture probes. Any of the aforementioned products can be released from the array, optionally amplified, and processed for library preparation as described herein to determine the location of the target nucleic acid in the biological sample.

### EMBODIMENTS

Embodiment 1 is a method for determining a location of a target RNA in a biological sample, the method comprising: a) hybridizing a primer to the target RNA in the biological sample; b) extending the primer using the target RNA as a template to provide a cDNA hybridized to the target RNA, thereby generating a cDNA:RNA duplex; c) incorporating at least three untemplated nucleotides at a 3′ end of the cDNA of the cDNA:RNA duplex; d) hybridizing a first adapter to the at least three untemplated nucleotides and extending the cDNA of the cDNA:RNA duplex using the first adapter as a template, thereby generating an extended cDNA:RNA duplex; c) contacting a transposome complex with the biological sample to insert a second adapter into the extended cDNA:RNA duplex, thereby generating a 5′ fragmented cDNA:RNA duplex; f) releasing the RNA from the 5′ fragmented cDNA:RNA duplex, thereby generating a 5′ cDNA molecule comprising a complement of the first adapter and the second adapter; g) hybridizing the first adapter of the 5′ cDNA molecule to a capture domain of a capture probe in an array comprising a plurality of capture probes, wherein the capture probe comprises: (i) a spatial barcode and (ii) a capture domain; and h) determining the sequence of (i) the spatial barcode, or a complement thereof, (ii) the 5′ cDNA molecule or a complement thereof, and using the determined sequences of (i) and (ii) to determine the location of the target RNA in the biological sample.

Embodiment 2 is the method of embodiment 1, wherein the biological sample is disposed on the array.

Embodiment 3 is the method of embodiment 1, wherein the biological sample is disposed on a first substrate.

Embodiment 4 is the method of embodiment 3, wherein the method further comprises aligning the first substrate comprising the biological sample with a second substrate comprising the array, such that at least a portion of the biological sample is aligned with at least a portion of the array.

Embodiment 5 is the method of any one of embodiments 1-4, wherein the method further comprises migrating the 5′ cDNA molecule from the biological sample to the array, and optionally, wherein the migrating comprises electrophoresis.

Embodiment 6 is the method of any one of embodiments 1-5, wherein the capture probe comprises one or more functional domains, a cleavage domain, a unique molecular identifier (UMI), or combinations thereof.

Embodiment 7 is the method of embodiment 6, wherein the one or more functional domains comprise a sequencing specific site or a primer binding site.

Embodiment 8 is the method of any one of embodiments 1-7, wherein the extending in step (b) comprises use of a reverse transcriptase.

Embodiment 9 is the method of any one of embodiments 1-8, wherein incorporating the at least three untemplated nucleotides comprises use of a reverse transcriptase.

Embodiment 10 is the method of any one of embodiments 1-8, wherein incorporating the at least three untemplated nucleotides comprises use of a terminal transferase.

Embodiment 11 is the method of embodiment 10, wherein the terminal transferase is a terminal deoxynucleotidyl transferase.

Embodiment 12 is the method of any one of embodiments 1-11, wherein the first adapter comprises RNA.

Embodiment 13 is the method of any one of embodiments 1-12, wherein the extending in step (d) comprises use of a polymerase.

Embodiment 14 is the method of embodiment 13, wherein the polymerase is a DNA polymerase, optionally an RNA dependent DNA polymerase.

Embodiment 15 is the method of any one of embodiments 1-14, wherein the at least three untemplated nucleotides comprise a homopolynucleotide sequence.

Embodiment 16 is the method of any one of embodiments 1-14, wherein the at least three untemplated nucleotides comprise a heteropolynucleotide sequence.

Embodiment 17 is the method of any one of embodiments 1-16, wherein the second adapter sequences comprise a functional domain.

Embodiment 18 is the method of embodiment 17, wherein the functional domain comprises a primer binding site.

Embodiment 19 is the method of any one of embodiments 1-18, wherein the array comprises one or more features.

Embodiment 20 is the method of embodiment 19, wherein the one or more features comprises a spot, a well, a post, a pit, a ridge, a divot, or a bead.

Embodiment 21 is the method of any one of embodiments 1-20, wherein the transposome complex comprises a transposase enzyme, a transposon sequence, and the second adapter; and optionally wherein the transposase enzyme is a Tn5 transposase enzyme, a Mu transposase enzyme, a Tn7 transposase enzyme, a Vibrio species transposase, or functional derivatives thereof.

Embodiment 22 is the method of embodiment 21, wherein the Tn5 transposase enzyme comprises a sequence that is at least 80% identical to SEQ ID NO: 1.

Embodiment 23 is the method of any one of embodiments 1-22, wherein releasing the RNA comprises denaturation.

Embodiment 24 is the method of embodiment 23, wherein denaturation comprises use of heat or potassium hydroxide.

Embodiment 25 is the method of any one of embodiments 1-22, wherein releasing the RNA comprise use of an RNase.

Embodiment 26 is the method of embodiment 25, wherein the RNase comprise one or more of RNase A, RNase C, RNase H, and RNase I, optionally, wherein the RNase H comprises one or both of RNase H1 and RNase H2.

Embodiment 27 is the method of any one of embodiments 1-26, wherein the second adapter is inserted at a 5′ end of the cDNA in the 5′ fragmented cDNA:RNA duplex.

Embodiment 28 is the method of any one of embodiments 1-27, wherein step (f) further comprises generating a 3′ fragmented cDNA:RNA duplex and one or more middle fragmented cDNA:RNA duplexes.

Embodiment 29 is the method of embodiment 28, further comprising a reverse transcription reaction to gap-fill the 3′ fragmented cDNA:RNA duplex and the one or more middle fragmented cDNA:RNA duplexes.

Embodiment 30 is the method of embodiment 28 or 29, further comprising releasing the RNA from the 3′ fragmented cDNA:RNA duplex and the one or more middle fragmented cDNA:RNA duplexes, thereby generating a 3′ cDNA molecule and one or more middle cDNA molecule(s), respectively.

Embodiment 31 is the method of any one of embodiments 28-30, wherein the array comprises a second plurality of capture probes, wherein a second capture probe of the second plurality of capture probes comprises: (i) a second spatial barcode and (ii) a second capture domain.

Embodiment 32 is the method of any one of embodiments 28-31, wherein the second adapter comprises a sequence complementary to the second capture domain.

Embodiment 33 is the method of any one of embodiments 28-32, further comprising hybridizing the second adapter of: the 3′ cDNA molecule to the second capture domain, and the one or more middle fragmented cDNA molecule(s) to different second capture domain(s).

Embodiment 34 is the method of any one of embodiments 31-33, further comprising determining the sequence of: (i) the second spatial barcode, or a complement thereof, and (ii) the 3′ cDNA molecule, or a complement thereof, and using the determined sequences of (i) and (ii) to determine the location of the target RNA in the biological sample.

Embodiment 35 is the method of any one of embodiments 31-34, further comprising determining the sequence of: (i) the second spatial barcode, or a complement thereof, and (ii) the one or more middle cDNA molecule(s), or complement(s) thereof, and using the determined sequences of (i) and (ii) to determine the location of the target RNA in the biological sample.

Embodiment 36 is the method of any one of embodiments 1-35, further comprising extending the capture probe using the 5′ cDNA molecule as a template.

Embodiment 37 is the method of any one of embodiments 1-36, further comprising extending the 5′ cDNA molecule using the capture probe as a template.

Embodiment 38 is the method of any one of embodiments 28-37, further comprising extending the second capture probe using the 3′ cDNA molecule and/or one of the one or more middle cDNA molecule(s) as a template.

Embodiment 39 is the method of any one of embodiments 28-38, further comprising extending the 3′ cDNA molecule and/or one of the one or more middle cDNA molecule(s) using the second capture probe as a template.

Embodiment 40 is the method of any one of embodiments 1-39, wherein the determining step comprises sequencing.

Embodiment 41 is the method of embodiment 40, wherein the sequencing comprises high-throughput sequencing.

Embodiment 42 is the method of any one of embodiments 1-41, wherein the method further comprises permeabilizing the biological sample.

Embodiment 43 is the method of embodiment 42, wherein the permeabilizing comprises use of a protease and/or detergent.

Embodiment 44 is the method of embodiment 43, wherein the protease comprises pepsin.

Embodiment 45 is the method of embodiment 43, wherein the protease comprises proteinase K.

Embodiment 46 is the method of any one of embodiments 1-45, wherein the biological sample is fixed.

Embodiment 47 is the method of embodiment 46, wherein the biological sample is methanol-fixed, acetone-fixed, paraformaldehyde-fixed, or is formalin-fixed paraffin-embedded (FFPE).

Embodiment 48 is the method of any one of embodiments 1-47, wherein the method further comprises staining the biological sample.

Embodiment 49 is the method of embodiment 48, wherein the staining comprises use of immunofluorescence, immunohistochemistry, and/or hematoxylin and/or eosin.

Embodiment 50 is the method of any one of embodiments 1-49, wherein the method further comprises imaging the biological sample.

Embodiment 51 is the method of any one of embodiments 1-50, wherein the biological sample is a tissue sample.

Embodiment 52 is the method of embodiment 51, wherein the tissue sample is a fixed tissue sample.

Embodiment 53 is the method of embodiment 52, wherein the fixed tissue sample is a methanol-fixed tissue sample, an acetone-fixed tissue sample, a paraformaldehyde tissue sample, or a formalin-fixed paraffin-embedded tissue sample.

Embodiment 54 is the method of embodiment 51, wherein the tissue sample is a fresh-frozen tissue sample.

Embodiment 55 is the method of any one of embodiments 1-50, wherein the biological sample is a tissue section.

Embodiment 56 is the method of embodiment 55, wherein the tissue section is a fixed tissue section.

Embodiment 57 is he method of embodiment 56, wherein the fixed tissue section is a methanol-fixed tissue section, an acetone-fixed tissue section, a paraformaldehyde tissue section, or a formalin-fixed paraffin-embedded tissue section.

Embodiment 58 is the method of embodiment 55, wherein the tissue section is a fresh-frozen tissue section.

Embodiment 59 is the method of any one of embodiments 1-58, wherein the primer hybridizes to a poly(A) sequence present in the target RNA.

Embodiment 60 is the method of embodiment 59, wherein the target RNA is mRNA.

Embodiment 61 is the method of any one of embodiments 1-60, further comprising use of a DNase.

Embodiment 62 is the method of any one of embodiments 1-61, wherein the first adapter comprises a template switch oligonucleotide.

Embodiment 63 is a kit comprising: a) an array comprising a plurality of capture probes, wherein a capture probe of the plurality of capture probes comprises: (i) a spatial barcode and (ii) a capture domain; b) a plurality of transposome complexes; c) a plurality of primers complementary to a plurality of target RNAs; d) a plurality of first adapters; and e) instructions for performing any of the methods of embodiments 1-62.

Embodiment 64 is the kit of embodiment 63, wherein the kit further comprises one or more permeabilization reagents.

Embodiment 65 is the kit of embodiment 64, wherein the one or more permeabilization reagents comprises one or more proteases, a DNase, an RNase, a lipase, a detergent, or combinations thereof.

Embodiment 66 is the kit of embodiment 65, wherein the one or more proteases comprise pepsin, proteinase K, or collagenase.

Embodiment 67 is the kit of any one of embodiments 63-66, wherein the capture probe further comprises one or more functional domains, a cleavage domain, a unique molecular identifier (UMI), or combinations thereof.

Embodiment 68 is the kit of embodiment 67, wherein the one or more functional domains comprises a primer binding site or a sequencing specific site.

Embodiment 69 is the kit of any one of embodiments 63-68, further comprising a polymerase.

Embodiment 70 is the kit of embodiment 69, wherein the polymerase comprises a reverse transcriptase and/or a DNA polymerase.

Embodiment 71 is the kit of any one of embodiments 63-70, wherein a transposome complex of the plurality of transposome complexes comprises a transposase enzyme, a transposon sequence, and a second adapter.

Embodiment 72 is the kit of embodiment 71, wherein the transposase enzyme is a Tn5 transposase enzyme, a Mu transposase enzyme, a Tn7 transposase enzyme, a Vibrio species transposase, or functional derivatives thereof.

Embodiment 73 is the kit of any one of embodiments 63-72, wherein a first adapter of the plurality of first adapters comprises a template switch oligonucleotide.

Embodiment 74 is a composition comprising: a) an array comprising a plurality of capture probes, wherein a capture probe of the plurality of capture probes comprises: (i) a spatial barcode and (ii) a capture domain; (b) a plurality of transposome complexes; (c) a plurality of primers complementary to a plurality of target RNAs; and (d) a plurality of first adapters.

Embodiment 75 is a composition comprising: an array comprising a plurality of capture probes, wherein a capture probe of the plurality of capture probes comprises: (i) a spatial barcode and (ii) a capture domain; and a plurality of cDNA:RNA duplexes.

Embodiment 76 is a composition comprising: (a) an array comprising a plurality of capture probes, wherein a capture probe of the plurality of capture probes comprises: (i) a spatial barcode and (ii) a capture domain; (b) a plurality of transposome complexes; and a plurality of 5′ end fragmented cDNA:RNA duplexes, a plurality of 3′ end fragmented cDNA:RNA duplexes, and/or one or more middle fragmented cDNA:RNA duplexes.

Embodiment 77 is a composition comprising: (a) an array comprising a plurality of capture probes, wherein a capture probe of the plurality of capture probes comprises: (i) a spatial barcode and (ii) a capture domain; and (b) a plurality of 5′ end fragmented cDNA:RNA duplexes, wherein the cDNA of the 5′ end fragmented cDNA:RNA duplex comprises a first adapter at the 3′ end.

Embodiment 78 is a composition comprising: (a) an array comprising a plurality of capture probes, wherein a capture probe of the plurality of capture probes comprises: (i) a spatial barcode and (ii) a capture domain; and (b) a plurality of gap-filled 3′ end fragmented cDNA:RNA duplexes and one or more gap-filled middle fragmented cDNA:RNA duplexes.

Embodiment 79 is a composition comprising: (a) an array comprising a plurality of capture probes, wherein a capture probe of the plurality of capture probes comprises: (i) a spatial barcode and (ii) a capture domain; (b) a plurality of extended 5′ end fragmented cDNA:RNA duplexes, a plurality of gap-filled 3′ end fragmented cDNA:RNA duplexes, and one or more gap-filled middle fragmented cDNA:RNA duplexes; and (c) an RNase.

Embodiment 80 is a composition comprising: (a) an array comprising a plurality of capture probes, wherein a capture probe of the plurality of capture probes comprises: (i) a spatial barcode and (ii) a capture domain; (b) a plurality of 5′ cDNA molecules comprising a first adapter sequence; (c) a plurality of 3′ cDNA molecules comprising a second adapter sequence; and (d) one or more gap-filled middle fragmented cDNA molecules comprising a second adapter sequence.

Embodiment 81 is a composition comprising: an array comprising: (i) a plurality of capture probes, wherein a capture probe of the plurality of capture probes comprises: (i) a spatial barcode and (ii) a capture domain; and (ii) a second plurality of capture probes, wherein a second capture probe of the second plurality of capture probes comprises: (i) a second spatial barcode and (ii) a second capture domain; and (b) one or more of: (i) a plurality of 5′ cDNA molecules comprising a first adapter sequence hybridized to the capture domain of the capture probe; (ii) a plurality of 3′ cDNA molecules comprising a second adapter sequence hybridized to a second capture domain of a second capture probe; and (iii) one or more gap-filled middle fragmented cDNA molecules comprising a second adapter sequence hybridized to a different second capture domain of a second capture probe.

Embodiment 82 is the composition of any one of embodiments 74-81, wherein the composition further comprises one or more permeabilization reagents.

Embodiment 83 is the composition of embodiment 82, wherein the one or more permeabilization reagents comprises one or more proteases, a DNase, an RNase, a lipase, a detergent, or combinations thereof.

Embodiment 84 is the composition of embodiment 83, wherein the one or more proteases comprise pepsin, proteinase K, and collagenase.

Embodiment 85 is the composition of any one of embodiments 74-84, wherein the capture probe further and/or the second capture probe comprises one or more functional domains, a cleavage domain, a unique molecular identifier (UMI), or combinations thereof.

Embodiment 86 is the composition of embodiment 85, wherein the one or more functional domains comprises a primer binding site or a sequencing specific site.

Embodiment 87 is the composition of any one of embodiments 74-86, further comprising a polymerase.

Embodiment 88 is the composition of embodiment 87, wherein the polymerase comprises a reverse transcriptase and/or a DNA polymerase.

Embodiment 89 is the composition of any one of embodiments 74-88, wherein a transposome complex of the plurality of transposome complexes comprises a transposase enzyme, a transposon sequence, and an adapter.

Embodiment 90 is the composition of embodiment 89, wherein the transposase enzyme is a Tn5 transposase enzyme, a Mu transposase enzyme, a Tn7 transposase enzyme, a Vibrio species transposase, or functional derivatives thereof.

Embodiment 91 is the composition of any one of embodiments 74-90, wherein a first adapter of the plurality of first adapters comprises a template switch oligonucleotide.

Embodiment 92 is a method for processing a target RNA in a biological sample, the method comprising: a) hybridizing a primer to the target RNA in the biological sample; b) extending the primer using the target RNA as a template to provide a cDNA hybridized to the target RNA, thereby generating a cDNA:RNA duplex; c) incorporating at least three untemplated nucleotides at a 3′ end of the cDNA of the cDNA:RNA duplex; d) hybridizing a first adapter to the at least three untemplated nucleotides and extending the cDNA of the cDNA:RNA duplex using the first adapter as a template, thereby generating an extended cDNA:RNA duplex; e) contacting a transposome complex with the biological sample to insert a second adapter into the extended cDNA:RNA duplex, thereby generating a 5′ fragmented cDNA:RNA duplex; and f) releasing the RNA from the 5′ fragmented cDNA:RNA duplex, thereby generating a 5′ cDNA molecule comprising a complement of the first adapter and the second adapter.

What is claimed is:

1. A method for determining a location of a target RNA in a biological sample, the method comprising:
a) hybridizing a primer to the target RNA in the biological sample;
b) extending the primer using the target RNA as a template to provide a cDNA hybridized to the target RNA, thereby generating a cDNA:RNA duplex;
c) incorporating at least three untemplated nucleotides at a 3′ end of the cDNA of the cDNA:RNA duplex;
d) hybridizing a first adapter to the at least three untemplated nucleotides and extending the cDNA of the cDNA:RNA duplex using the first adapter as a template, thereby generating an extended cDNA:RNA duplex;
e) contacting a transposome complex with the biological sample to insert a second adapter into the extended cDNA:RNA duplex, thereby generating a 5′ fragmented cDNA:RNA duplex;
f) releasing the RNA from the 5′ fragmented cDNA:RNA duplex, thereby generating a 5′ cDNA molecule comprising (i) a complement of the first adapter, and (ii) the second adapter;
g) hybridizing the first adapter of the 5′ cDNA molecule to a capture domain of a capture probe in an array comprising a plurality of capture probes, wherein the capture probe comprises: (i) a spatial barcode and (ii) a capture domain; and
h) determining the sequence of (i) the spatial barcode, or a complement thereof, (ii) the 5′ cDNA molecule or a complement thereof, and using the determined sequences of (i) and (ii) to determine the location of the target RNA in the biological sample.

2. The method of claim 1, wherein the biological sample is disposed on the array or wherein the biological sample is disposed on a first substrate.

3. The method of claim 2, wherein when the biological sample is disposed on a first substrate, the method further comprises aligning the first substrate comprising the biological sample with a second substrate comprising the array, such that at least a portion of the biological sample is aligned with at least a portion of the array, and migrating the 5′ cDNA molecule from the biological sample to the array, optionally wherein the migrating comprises electrophoresis.

4. The method of claim 1, wherein the extending in step (b) comprises use of a reverse transcriptase, wherein incorporating the at least three untemplated nucleotides comprises use of the reverse transcriptase.

5. The method claim 1, wherein incorporating the at least three untemplated nucleotides comprises use of a terminal transferase, optionally wherein the terminal transferase is a terminal deoxynucleotidyl transferase.

6. The method of claim 1, wherein the first adapter comprises RNA.

7. The method of claim 1, wherein the at least three untemplated nucleotides comprise a homopolynucleotide sequence or a heteropolynucleotide sequence.

8. The method of claim 1, wherein the second adapter sequences comprise a functional domain, optionally wherein the functional domain comprises a primer binding site.

9. The method of claim 1, wherein the transposome complex comprises a transposase enzyme, a transposon sequence, and the second adapter; and optionally wherein the transposase enzyme is a Tn5 transposase enzyme, a Mu transposase enzyme, a Tn7 transposase enzyme, a Vibrio species transposase, or functional derivatives thereof.

10. The method of claim 1, wherein releasing the RNA comprises use of heat, potassium hydroxide, or an RNase, optionally wherein the RNase comprise one or more of RNase A, RNase C, RNase H, and RNase I.

11. The method of claim 1, wherein the second adapter is inserted at a 5′ end of the cDNA in the 5′ fragmented cDNA:RNA duplex.

12. The method of claim 1, wherein step (e) further comprises generating a 3′ fragmented cDNA:RNA duplex and one or more middle fragmented cDNA:RNA duplexes, the method further comprising a reverse transcription reaction to gap-fill the 3′ fragmented cDNA:RNA duplex and the one or more middle fragmented cDNA:RNA duplexes.

13. The method of claim 12, further comprising releasing RNA from the 3′ fragmented cDNA:RNA duplex and the one or more middle fragmented cDNA:RNA duplexes, thereby generating a 3′ cDNA molecule and one or more middle cDNA molecule(s), respectively.

14. The method of claim 13, wherein the array comprises a second plurality of capture probes, wherein a second capture probe of the second plurality of capture probes comprises: (i) a second spatial barcode and (ii) a second capture domain; wherein the second adapter comprises a sequence complementary to the second capture domain.

15. The method of claim 14, further comprising hybridizing the second adapter of the 3′ cDNA molecule to the second capture domain, and determining the sequence of: (i) the second spatial barcode, or a complement thereof, and (ii) the 3′ cDNA molecule, or a complement thereof, and using the determined sequences of (i) and (ii) to determine the location of the target RNA in the biological sample.

16. The method of claim 1, further comprising extending the capture probe using the 5′ cDNA molecule as a template and/or extending the 5′ cDNA molecule using the capture probe as a template.

17. The method of claim 1, wherein the determining step comprises sequencing.

18. The method of claim 1, wherein the method further comprises permeabilizing, staining, and/or imaging the biological sample, and wherein the biological sample is a tissue section, optionally a fresh-frozen tissue section or a fixed tissue section.

19. The method of claim 1, wherein the target RNA is mRNA.

20. A method for processing a target RNA in a biological sample, the method comprising:
a) hybridizing a primer to the target RNA in the biological sample;
b) extending the primer using the target RNA as a template to provide a cDNA hybridized to the target RNA, thereby generating a cDNA:RNA duplex;
c) incorporating at least three untemplated nucleotides at a 3′ end of the cDNA of the cDNA:RNA duplex;
d) hybridizing a first adapter to the at least three untemplated nucleotides and extending the cDNA of the cDNA:RNA duplex using the first adapter as a template, thereby generating an extended cDNA:RNA duplex;
e) contacting a transposome complex with the biological sample to insert a second adapter into the extended cDNA:RNA duplex, thereby generating a 5′ fragmented cDNA:RNA duplex; and
f) releasing the RNA from the 5′ fragmented cDNA:RNA duplex, thereby generating a 5′ cDNA molecule comprising a complement of the first adapter and the second adapter.


SEQUENCE LISTING (33 sequences)

SEQ ID NO: 1 - protein, 476 aa, Escherichia coli
MITSALHRAADWAKSVFSSAALGDPRRTARLVNVAAQLAKYSGKSITISSEGSEAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTLPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 2 - DNA, 19 bp, synthetic construct
  source 1..19: mol_type = other DNA
ctgtctcttatacacatct

SEQ ID NO: 3 - protein, 662 aa, Bacteriophage mu
MKEWYTAKELLGLAGLPKQATNITRKAQREGWEFRQVAGTKGVSFEFNIKSFPVALRAEILLQQGRIETSQGYFEIARPTLEAHDYDREALWSKWDNASDSQRRLAEKWLPAVQAADEMLNQGISTKTAFATVAGHYQVSASTLRDKYYQVQKFAKPDWAAALVDGRGASRRNVHKSEFDEDAWQFLIADYLRPEKPAFRKCYERLELAAREHGWSIPSRATAFRRIQQLDEAMVVACREGEHALMHLIPAQQRTVEHLDAMQWINGDGYLHNVFVRWFNGDVIRPKTWFWQDVKTRKILGWRCDVSENIDSIRLSFMDVVTRYGIPEDFHITIDNTRGAANKWLTGGAPNRYRFKVKEDDPKGLFLLMGAKMHWTSVVAGKGWGQAKPVERAFGVGGLEEYVDKHPALAGAYTGPNPQAKPDNYGDRAVDAELFLKTLAEGVAMFNARTGRETEMCGGKLSFDDVFEREYARTIVRKPTEEQKRMLLLPAEAVNVSRKGEFALKVGGSLKGAKNVYYNMALMNAGVKKVVVRFDPQQLHSTVYCYTLDGRFICEAECLAPVAFNDAAAGREYRRRQKQLKSATKAAIKAQKQMDALEVAELLPQIAEPEAPESRIVGIFRPSGNTERVKNQERDDEYETERDEYLNHSLDILEQNRRKKAI

SEQ ID NO: 4 - DNA, 27 bp, synthetic construct
  source 1..27: mol_type = other DNA
tgaagcggcgcacgaaaaacgcgaaag

SEQ ID NO: 5 - DNA, 23 bp, synthetic construct
  source 1..23: mol_type = other DNA
gcgtttcacgataaatgcgaaaa

SEQ ID NO: 6 - DNA, 23 bp, synthetic construct
  source 1..23: mol_type = other DNA
ctgtttcatttgaagcgcgaaag

SEQ ID NO: 7 - DNA, 27 bp, synthetic construct
  source 1..27: mol_type = other DNA
tgtattgattcacttgaagtacgaaaa

SEQ ID NO: 8 - DNA, 23 bp, synthetic construct
  source 1..23: mol_type = other DNA
ccttaatcaatgaaacgcgaaag

SEQ ID NO: 9 - DNA, 23 bp, synthetic construct
  source 1..23: mol_type = other DNA
ttgtttcattgaaaatacgaaaa

SEQ ID NO: 10 - protein, 458 aa, synthetic construct
MTHSDAKLWAQEQFGQAQLKDPRRTQRLISLATSIANQPGVSVAKLPFSPADMEGAYRFIRNENINAEDIAEAGFQSTVSRANEHKELLALEDTTTLSFPHRSIKEELGHTNQGDRTRALHVHSTLLFAPQSQTIVGLIEQQRWSEDITKRGQKHQHATRPYKEKESYKWEQASRRVVERLGDKMLDVISVCDREADLFEYLTYKRQHQQRFVVRSMQSRCLEEHAQKLYDYAQALPSVETKALTIPQKGGRKARNVKLDVKYGQVTLKAPANKKEHAGIPVYYVGCLEQGTSKDLAWHLLTSEPINNVDDAMRIIGYYERRWLIEDFHKVWKSEGTDVESLRLQSKDNLERLSVIYAFVATRLLALRFMKEVDELTKESCEKVLGQKAWKLLWLKLESKTLPKEVPDMGWAYKNLAKLGGWKDTKRTGRASIKVLWEGWFKLQTILEGYELAMSLDH

SEQ ID NO: 11 - DNA, 19 bp, synthetic construct
  source 1..19: mol_type = other DNA
ctgtctcttgatcacaagt

SEQ ID NO: 12 - DNA, 19 bp, synthetic construct
  source 1..19: mol_type = other DNA
agatgtgatcaagagacag

SEQ ID NO: 13 - DNA, 19 bp, synthetic construct
  source 1..19: mol_type = other DNA
ctgtctcttgatcacatct

SEQ ID NO: 14 - DNA, 92 bp, unidentified
  source 1..92: mol_type = other DNA
aagcagtggtatcaacgcagagtacatgggnnnnnnnnnnnnnnnnnnnnnnnnnnnnnnnbaaaaaaaaaaaaaaaaaaaaaaaaaaaaaa

SEQ ID NO: 15 - DNA, 63 bp, unidentified
  source 1..63: mol_type = other DNA
tvnnnnnnnnnnnnnnnnnnnnnnnnnnnnnnncccatgtactctgcgttgataccactgctt

SEQ ID NO: 16 - DNA, 42 bp, unidentified
  source 1..42: mol_type = other DNA
aagcagtggtatcaacgcagagtacatgggnnnnnnnnnnnn

SEQ ID NO: 17 - DNA, 85 bp, unidentified
  source 1..85: mol_type = other DNA
gtctcgtgggctcggagatgtgtataagagacagnnnnnnnnnnnnnnnnnnnnncccatgtactctgcgttgataccactgctt

SEQ ID NO: 18 - DNA, 86 bp, unidentified
  source 1..86: mol_type = other DNA
gtctcgtgggctcggagatgtgtataagagacagnnnnnnnnnnnnnnnnnnnnnbaaaaaaaaaaaaaaaaaaaaaaaaaaaaaa

SEQ ID NO: 19 - DNA, 42 bp, unidentified
  source 1..42: mol_type = other DNA
ttttttttttttttttttttttttttttttvnnnnnnnnnnn

SEQ ID NO: 20 - DNA, 55 bp, unidentified
  source 1..55: mol_type = other DNA
gtctcgtgggctcggagatgtgtataagagacagnnnnnnnnnnnnnnnnnnnnn

SEQ ID NO: 21 - DNA, 40 bp, unidentified
  source 1..40: mol_type = other DNA
tctacacatattctctgtcnnnnnnnnnnnnnnnnnnnnn

SEQ ID NO: 22 - DNA, 22 bp, unidentified
  source 1..22: mol_type = other DNA
ctacacgacgctcttccgatct

SEQ ID NO: 23 - DNA, 85 bp, unidentified
  source 1..85: mol_type = other DNA
aagcagtggtatcaacgcagagtacatgggnnnnnnnnnnnnnnnnnnnnnctgtctcttatacacatctccgagcccacgagac

SEQ ID NO: 24 - DNA, 85 bp, unidentified; identical to SEQ ID NO: 17
  source 1..85: mol_type = other DNA

SEQ ID NO: 25 - DNA, 86 bp, unidentified
  source 1..86: mol_type = other DNA
cagagcacccgagcctctacacatattctctgtcnnnnnnnnnnnnnnnnnnnnnbaaaaaaaaaaaaaaaaaaaaaaaaaaaaaa

SEQ ID NO: 26 - DNA, 86 bp, unidentified
  source 1..86: mol_type = other DNA
ttttttttttttttttttttttttttttttvnnnnnnnnnnnnnnnnnnnnngacagagaatatgtgtagaggctcgggtgctctg

SEQ ID NO: 27 - DNA, 98 bp, unidentified
  source 1..98: mol_type = other DNA
cagagcacccgagccagatgtgtataagagacagnnnnnnnnnnnnnnnnnnnnnnnnnnnnnnctgtctcttatacacatctccgagcccacgagac

SEQ ID NO: 28 - DNA, 98 bp, unidentified
  source 1..98: mol_type = other DNA
gtctcgtgggctcggagatgtgtataagagacagnnnnnnnnnnnnnnnnnnnnnnnnnnnnnnctgtctcttatacacatctggctcgggtgctctg

SEQ ID NO: 29 - DNA, 19 bp, unidentified
  source 1..19: mol_type = other DNA
ctgtctcttatacacatct

SEQ ID NO: 30 - DNA, 19 bp, unidentified
  source 1..19: mol_type = other DNA
ctgtctcttatacacatct

SEQ ID NO: 31 - DNA, 19 bp, unidentified
  source 1..19: mol_type = other DNA
ctgtctcttatacacatct

SEQ ID NO: 32 - DNA, 34 bp, unidentified
  source 1..34: mol_type = other DNA
gacagagaatatgtgtagaggctcgggtgctctg

SEQ ID NO: 33 - DNA, 26 bp, unidentified
  source 1..26: mol_type = other DNA
agcagtggtatcaacgcagagtacat